(19) United States
(12) Patent Application Publication
(10) Pub. No.: US 2017/0166875 A1
(43) Pub. Date: Jun. 15, 2017

(54) METHODS RELATING TO NUCLEIC ACID SEQUENCE EDITING

(71) Applicant: UNIVERSITY OF WASHINGTON, Seattle, WA (US)

(72) Inventors: Nancy Maizels, Seattle, WA (US); Luther Davis, Seattle, WA (US); Charlotte A. James, Seattle, WA (US); Yinbo Zhang, Seattle, WA (US)

(73) Assignee: UNIVERSITY OF WASHINGTON, Seattle, WA (US)

(21) Appl. No.: 15/376,876

(22) Filed: Dec. 13, 2016

Related U.S. Application Data
(60) Provisional application No. 62/267,196, filed on Dec. 14, 2015.

(51) Int. Cl. C12N 9/22; C12N 15/85; C12P 19/34

(52) U.S. Cl. CPC C12N 9/22; C12P 19/34; C12Y 301/00; C12N 15/85; C12N 2800/80

(57) ABSTRACT

The technology described herein is directed to engineered endonucleases whose activity is restricted to certain phases of the cell cycle. Provided herein are compositions and methods relating to such engineered endonucleases.

### CROSS-REFERENCE TO RELATED APPLICATIONS

This application claims benefit under 35 U.S.C. §119(e) of U.S. Provisional Application No. 62/267,196 filed Dec. 14, 2015, the contents of which are incorporated herein by reference in their entirety.

### GOVERNMENT SUPPORT

This invention was made with government support under Grant No. R01 CA183967 awarded by the National Institutes of Health. The government has certain rights in the invention.

### SEQUENCE LISTING

Technical Field

The technology described herein relates to methods for optimizing nucleic acid sequence editing, e.g., by preferentially using either homology-directed repair (HDR) or mutagenic end-joining (mutEJ) to repair endonuclease-generated nicks or double strand breaks (DSBs).

Background

CRISPR/Cas and other RNA-guided endonucleases now make essentially all sites in all genomes accessible to genome engineering. The current challenge is to optimize the outcomes of targeted DNA cleavage, maximizing efficiency while minimizing off-target damage. Targeted DSBs are accompanied by extensive damage, evident as mutEJ and less frequently as translocations at the target and at off-target sites. Targeted nicks offer clear advantages for genome engineering, as mutEJ frequencies are considerably reduced as assayed using reporters and by genome-wide approaches.

The choice between mutEJ and HDR depends on the phase of the cell cycle. One very active component of mutEJ is the non-homologous end-joining (NHEJ), pathway, which is active throughout cell cycle but especially in G1 phase; while repair by HDR is most active in S phase (Karanam et al. 2012 Mol Cell 47:320-329).

### SUMMARY

As described herein, the inventors have demonstrated that this temporal regulation does affect the outcome of genome engineering initiated by targeted DSBs or targeted nicks, and accordingly provide compositions and methods that permit the user to utilize temporal regulation of endonuclease activity to control the outcome of genome engineering techniques. Described herein is the utilization of tags derived from cell cycle regulators to restrict nuclear activity of Cas9^{D10A} (nicks) or Cas9 (DSBs) to G1 or S-G2/M phases. These tags, e.g., those derived from the CDT1 and Geminin cell cycle regulators, specify nuclear degradation of the fused protein outside G1 or S-G2/M phases, respectively. By generating nicks using endonucleases with the foregoing tags, it is demonstrated herein that nicks initiate homology-directed repair (HDR) much more efficiently in G1 phase than in S-G2/M phases, while DSBs initiate HDR more efficiently in S-G2/M phase than in G1 phase.

The activity of the tagged proteins was tested using the Traffic Light reporter, which scores HDR events as expression of GFP, and mutEJ events as expression of mCherry. Targeted nicks were demonstrated to initiate HDR by a single-stranded oligonucleotide (SSO) donor most efficiently in G1 phase, and mutEJ occurs with a much lower frequency than with HDR. The methods and compositions described herein improve the efficiency and safety of targeted genome engineering by restricting endonuclease activity to particular phases of the cell cycle and thereby permitting preferential use of HDR or mutEJ as desired.

It is demonstrated herein that homology-directed repair (HDR) reaches frequencies of over 20% at nicks initiated in G1 phase, using single-stranded oligonucleotide (SSO) donors and in cells in which a very efficient alternative HDR pathway has been activated by downregulation of canonical HDR. Relatively little mutagenic end-joining (mutEJ) accompanies HDR at nicks. It is further demonstrated that SSO donors support high frequencies of HDR at DSBs, and that donor structure does not affect frequencies of mutEJ. Using SSO donors, G1-phase nicks and S-phase DSBs initiated comparably high levels of HDR, but the ratio of HDR:mutEJ was approximately 20:1 at G1 phase nicks, and 2:1 at S phase DSBs. Thus, G1 phase nicks offer a safer approach to gene correction and engineering than do S phase DSBs. Cell cycle-restricted derivatives of Cas9^{D10A} and Cas9 and other endonucleases that target DNA nicks or DSBs are of considerable utility in gene correction and genome engineering.

In one aspect of any of the embodiments, described herein is an engineered endonuclease comprising an endonuclease polypeptide and a cell cycle-dependent nuclear destruction tag. In some embodiments of any of the aspects, the endonuclease polypeptide comprises a sequence-specific endonuclease. In some embodiments of any of the aspects, the endonuclease polypeptide comprises an endonuclease selected from the group consisting of: Cas9; a Cas9-derived nuclease; Cas9^{D10A}; a Cas9 nickase variant; a TALEN; a ZFN; Cpf1; a nuclease comprising a FokI cleavage domain; a RNA-guided engineered nuclease; and a homing endonuclease.

In some embodiments of any of the aspects, the cell cycle-dependent nuclear destruction tag comprises a sequence found in a protein selected from the group consisting of GEM; CDT1; Orc1; Cdc25A; Cyclin A; Cyclin B1; Securin; Plk1; Cdc6; Cyclin E; c-Jun; c-Myc; and RAG-2. In some embodiments of any of the aspects, the cell cycle-dependent nuclear destruction tag comprises a Geminin (GEM) or chromatin licensing and DNA replication factor (CDT1) cell cycle-dependent nuclear destruction tag. In some embodiments of any of the aspects, the cell cycle-dependent nuclear destruction tag is selected from SEQ ID NO: 4, SEQ ID NO: 6, or SEQ ID NOs: 8-12. In some embodiments of any of the aspects, the cell cycle-dependent nuclear destruction tag is a sequence corresponding to a sequence selected from SEQ ID NO: 4, SEQ ID NO: 6, or SEQ ID NOs: 8-12.

In some embodiments of any of the aspects, the tag is located at the C-terminus of the endonuclease. In some embodiments of any of the aspects, the engineered endonuclease further comprises a linker sequence between the endonuclease polypeptide and cell cycle-dependent nuclear destruction tag. In some embodiments of any of the aspects, the linker sequence comprises the sequence GGGGS (SEQ ID NO: 2).

In one aspect of any of the embodiments, described herein is an isolated nucleic acid molecule encoding an engineered endonuclease comprising an endonuclease polypeptide and a cell cycle-dependent nuclear destruction tag. In one aspect of any of the embodiments, described herein is a vector comprising an isolated nucleic acid molecule encoding an engineered endonuclease comprising an endonuclease polypeptide and a cell cycle-dependent nuclear destruction tag.

In one aspect of any of the embodiments, described herein is a composition comprising: an engineered endonuclease comprising an endonuclease polypeptide and a cell cycle-dependent nuclear destruction tag and a donor nucleic acid sequence. In some embodiments of any of the aspects, the engineered endonuclease comprises a Cas9 or Cas9-derived endonuclease polypeptide and the composition further comprises one or more crRNA, tracrRNA, or sgRNA molecules.

In one aspect of any of the embodiments, described herein is a method of modifying the sequence of a target nucleic acid molecule, the method comprising contacting the target nucleic acid molecule with an engineered endonuclease comprising an endonuclease polypeptide and a cell cycle-dependent nuclear destruction tag. In some embodiments of any of the aspects, the target nucleic acid molecule is further contacted with a donor nucleic acid sequence. In some embodiments of any of the aspects, the engineered endonuclease comprises a Cas9 or Cas9-derived endonuclease polypeptide and the method further comprises contacting the target nucleic acid molecule with one or more crRNA, tracrRNA, or sgRNA molecules. In some embodiments of any of the aspects, the engineered endonuclease comprises an endonuclease polypeptide and a G1-restricting cell cycle-dependent nuclear destruction tag and the modification thereby occurs via homology-directed repair. In some embodiments of any of the aspects, the G1-restricting cell cycle-dependent nuclear destruction tag is a CDT1 cell cycle-dependent nuclear destruction tag. In some embodiments of any of the aspects, the engineered endonuclease comprises an endonuclease polypeptide and a S-G2/M-restricting cell cycle-dependent nuclear destruction tag and the modification thereby occurs via non-homologous end-joining (NHEJ) or mutagenic end-joining (mutEJ).

### BRIEF DESCRIPTION OF THE DRAWINGS

FIG. 1 depicts a schematic of the reporter used for repair analysis. HDR events will repair GFP gene and mutEJ events will move mCherry into an open reading frame. Further details of the reporter construction can be found, e.g., in Certo et al., 2011 and Davis et al, 2014.

FIG. 2 demonstrates the cell cycle stabilization of Cas9 or Cas9^{D10A} endonucleases tagged with CDT1 or Geminin (GEM). Depicted are graphs demonstrating that Cas9^{D10A}-mKO2-CDT1 displays nuclear stabilization in G1 (left panel) and that Cas9^{D10A}-mAG-GEM displays nuclear stabilization in S-G2/M (right panel). Cells were transfected with fluorecently tagged Cas9-CDT1 or Cas9-GEM. Among the cells that were positive for the presence of the tagged endonuclease, the relative distribution of cell cycle phases are shown above. G1 cells are to the left of the bold vertical line, while S-G2/M cells are to the right of the bold vertical line.

FIGS. 3A-3B demonstrate HDR and mutEJ frequencies at nicks and DSBs generated with cell cycle regulated Cas9 endonucleases. The figure presents repair frequencies among transfected cells following transfection with cell cycle tagged or untagged Cas9 or Cas9^{D10A} in cells provided with a SSO donor (mean±SEM, n=3). Nicks were generated in cells expressing BRC3, to stimulate alternative HDR. FIG. 3A depicts HDR frequencies. FIG. 3B depicts mutEJ frequencies. **=p≦0.001, *=p≦0.05, n.s.=not significant. See also, Tables 6 and 8.

FIG. 4 demonstrates canonical HDR and mutEJ frequencies at nicks generated with cell cycle regulated Cas9^{D10A} endonucleases. The figure presents repair frequencies among transfected cells following transfection with cell cycle tagged or untagged Cas9^{D10A} in cells provided with a SSO donor (mean±SEM, n=3). *=p≦0.05, n.s.=not significant. Cells were not expressing BRC3 peptide, in contrast to results shown in FIG. 3, so comparison of FIG. 3 and FIG. 4 show that expression of BRC3 peptide stimulates HDR at nicks by SSO donors. (See also, Table 5).

FIGS. 5A-5D demonstrate cell cycle-specific expression of Cas9^{D10A} in HEK 293T cells. Graphs in FIG. 5A-5B depict the percentage of cells (whole population and mAG+ cells, solid black or grey bars and solid or patterned bars, respectively) in G1 or S-G2/M phases. C and D, the percentage of cells (whole population and mKO2+ cells, solid black or grey bars and solid or patterned bars, respectively) in G1 or S-G2/M phases.

FIGS. 6A-6D depict histograms showing the cell number relative to DNA content. The transfecting DNA dose represents the amount of DNA (mAG-GEM or mKO2-CDT1 alone or fused to Cas9^{D10A}) per cell (number of cells plated 16 hours before the transfection).

FIG. 7 compares HDR and mut-EJ efficiency at nicks induced by CDT1- or GEM-tagged Cas9^{D10A}. The transfecting DNA dose represents the amount of DNA encoding CDT1- or GEM-tagged Cas9^{D10A} per cell (number of cells plated 16 hours before the transfection). The HDR (upper panel) and mut-EJ (lower panel) events at the Traffic Light reporter were measured as percentage of GFP+ and mCherry+ cells, respectively.

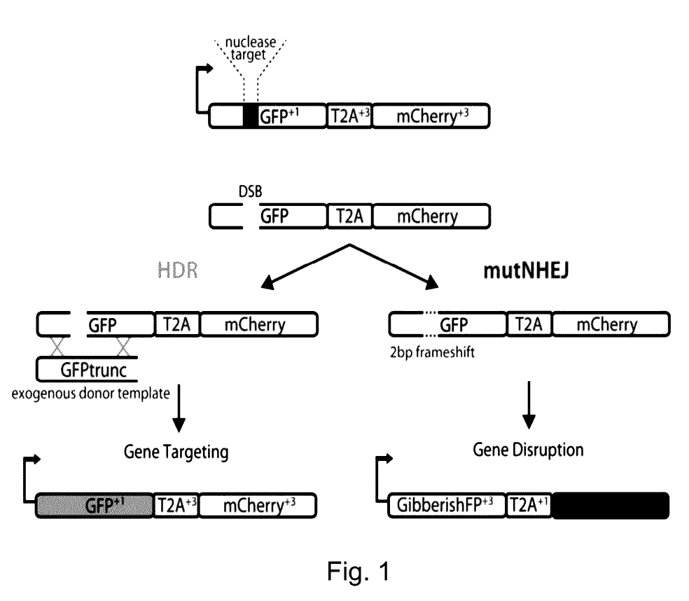

### DETAILED DESCRIPTION

The inventors have designed and demonstrated a strategy for engineering endonucleases which are active only during the desired portion of the cell cycle. Such temporal control of the endonuclease activity allows the user to more precisely control the endonuclease's activity, e.g., during gene editing or genome engineering.

In one aspect, provided herein is an engineered endonuclease comprising 1) an endonuclease polypeptide and 2) a cell cycle-dependent nuclear destruction tag. As used herein, “endonuclease” refers to an enzyme capable of cleaving the phosphodiester bonds between the nucleotide subunits of nucleic acids within a polynucleotide, e.g., cleaving a phosphodiester bond that is not either the 5′ or 3′ most bond present in the polynucleotide. In some embodiments of any of the aspects described herein, the endonuclease can generate nicks. In some embodiments of any of the aspects described herein, the endonuclease can generate double-strand breaks (DSBs).

In some embodiments of any of the aspects described herein, the endonuclease polypeptide can be and/or comprise a sequence-specific endonuclease. A sequence-specific endonuclease is an endonuclease which demonstrates specificity for specific sequences, e.g., the endonuclease will preferentially cut at, or at a predictable distance from, a given specific sequence and/or consensus sequence.

In some embodiments of any of the aspects described herein, the endonuclease polypeptide can be and/or comprise a programmable endonuclease. As used herein “programmable nuclease” refers to a nuclease that has been engineered to create a DSB or nick at a nucleic acid sequence that the native nuclease would not act upon, e.g. the sequence specificity of the nuclease has been altered. For example, Cas9-derived nucleases and nickases are targeted by means of guide nucleic acid molecules, with which the nuclease forms a complex. The guide RNAs can be engineered to hybridize specifically to a desired target nucleic acid sequence (or a flanking sequence). By way of further non-limiting example, zinc finger nucleases can be targeted by a combinatorial assembly of multiple zinc finger domains with known DNA triplet specificities. Methods of engineering nucleases to achieve a desired sequence specifity are known in the art and are described, e.g., in Kim and Kim. Nature Reviews Genetics 2014 15:321-334; Kim et al. Genome Res. 2012 22:1327-1333; Belhaj et al. Plant Methods 2013 9:39; Urnov et al. Nat Rev Genet 2010 11:636-646; Bogdanove et al. Science 2011 333:1843-6; Jinek et al. Science 2012 337:816-821; Silva et al. Curr Gene Ther 2011 11:11-27; Ran et al. Cell 2013 154:1380-9; Carlson et al. PNAS 212 109:17382-7, Guerts et al. Science 2009 325:433-3; Takasu et al. Insect Biochem Mol Biol 2010 40:759-765; and Watanabe et al. Nat. Commun. 2012 3; each of which is incorporated by reference herein in its entirety.

Non-limiting examples of sequence-specific endonucleases and/or programmable endonucleases can include Cas9; a Cas9-derived nuclease; Cas9^{D10A}; a Cas9 nickase variant; a TALEN; a ZFN; Cpf1; a nuclease comprising a FokI cleavage domain; a RNA-guided engineered nuclease; and a homing endonuclease.

The engineered endonucleases described herein are active only during a portion of the cell cycle due to inclusion of a cell cycle-dependent nuclear destruction tag. As used herein, “cell cycle-dependent nuclear destruction tag” refers to a polypeptide sequence which, when present in the nucleus, is recognized by a cell and targeted for destruction during only a portion of the cell cycle, e.g., by protease activity. When the cell cycle-dependent nuclear destruction tag is included in a larger polypeptide, the larger polypeptide will be destroyed along with the cell cycle-dependent nuclear destruction tag.

Provided herein are numerous examples of cell cycle-dependent nuclear destruction tags. For example, the polypeptides GEM; CDT1; Orc1; Cdc25A; Cyclin A; Cyclin B1; Securin; Plk1; Cdc6; Cyclin E; c-Jun; c-Myc; and RAG-2 are known to be regulated by cell cycle-dependent nuclear destruction and cell cycle-dependent nuclear destruction tags can be tags obtained from the sequences of these polypeptides or homologs and/or gene family members thereof. In some embodiments of any of the aspects described herein, the cell cycle-dependent nuclear destruction tag comprises a sequence found in a protein selected from the group consisting of GEM; CDT1; Orc1; Cdc25A; Cyclin A; Cyclin B1; Securin; Plk1; Cdc6; Cyclin E; c-Jun; c-Myc; and RAG-2. In some embodiments of any of the aspects described herein, the cell cycle-dependent nuclear destruction tag comprises a sequence found in a bacterial transposase. Sequences for the foregoing proteins are known in the art for a number of species. For example, human sequences are available in the NCBI database for, e.g., human GEM (NCBI Ref Seq: 51053); human CDT1 (NCBI Ref Seq: 81620); human Orc1 (NCBI Ref Seq: 4998); human Cdc25A (NCBI Ref Seq: 993); human Cyclin A (NCBI Ref Seq: 890); human Cyclin B1 (NCBI Ref Seq: 891); human Securin (NCBI Ref Seq: 9232); human Plk1 (NCBI Ref Seq: 5347); human Cdc6 (NCBI Ref Seq: 990); human Cyclin E (NCBI Ref Seq: 898); human c-Jun (NCBI Ref Seq: 3725); human c-Myc (NCBI Ref Seq: 4609); and human RAG-2 (NCBI Ref Seq: 5897).

In some embodiments of any of the aspects described herein, the cell cycle-dependent nuclear destruction tag can comprise a Geminin (GEM) or chromatin licensing and DNA replication factor (CDT1) cell cycle-dependent nuclear destruction tag. Exemplary human-origin cell cycle-dependent nuclear destruction tags from Geminin (GEM) and chromatin licensing and DNA replication factor (CDT1) include polypeptides having the sequence of SEQ ID NO: 4 or SEQ ID NO: 6 (See Table 11).

The cell cycle-dependent nuclear destruction tags described herein can be further characterized by the phase(s) of the cell cycle to which they restrict polypeptide stability. For example, a given cell cycle-dependent nuclear destruction tag can be targeted for destruction in S phase, G2 phase and M phase, resulting in the tag and any linked polypeptide being active in the nucleus only during G1 phase. Such a tag would be referred to as a G1-restricting cell cycle-dependent nuclear destruction tag because its stability in the nucleus (and the expression, stability and/or activity of any linked polypeptides) is restricted to G1 phase. A cell cycle-dependent nuclear destruction tag can be restricting for 1, 2, or 3 phases of the cell cycle, in any combination. For example a cell cycle-dependent nuclear destruction tag can be G1-restricting, S-restricting, G2-restricting, M-restricting or any combination thereof.

By way of non-limiting example, G1-restricting cell cycle-dependent nuclear destruction tags can include CDT1 cell cycle-dependent nuclear destruction tags, e.g., tags comprising the sequence of SEQ ID NO: 4. Additional CDT1 cell cycle-dependent nuclear destruction tags can include a sequence corresponding to amino acid residues 30-120, 30-546, 30-189, 30-100, 1-546, 1-189, and 1-100 of the polypeptide encoded by the nucleic acid sequence of SEQ ID NO: 3 (see, e.g, Sakaue-Sawano et al. 2008 Cell 132:487-498; which is incorporated by reference herein in its entirety, for further discussion). In some embodiments of any of the aspects described herein, a CDT1 cell cycle-dependent nuclear destruction tag can comprise a sequence of SEQ ID NO: 4 or amino acid residues 30-120, 30-546, 30-189, 30-100, 1-546, 1-189, or 1-100 of the polypeptide encoded by the nucleic acid sequence of SEQ ID NO: 3. In some embodiments of any of the aspects described herein, a CDT1 cell cycle-dependent nuclear destruction tag can consist of a sequence of SEQ ID NO: 4 or amino acid residues 30-120, 30-546, 30-189, 30-100, 1-546, 1-189, or 1-100 of the polypeptide encoded by the nucleic acid sequence of SEQ ID NO: 3.

By way of further non-limiting example, S-G2/M-restricting cell cycle-dependent nuclear destruction tags can include GEM cell cycle-dependent nuclear destruction tags, e.g., tags comprising the polypeptide sequence corresponding to SEQ ID NO: 6. Additional GEM cell cycle-dependent nuclear destruction tags can include sequences comprising amino acid residues 1-110, 1-60, 1-209, and 20-110 of the polypeptide encoded by the nucleic acid sequence of SEQ ID NO: 5 (see, e.g, Sakaue-Sawano et al. 2008 Cell 132:487-498; which is incorporated by reference herein in its entirety, for further discussion). In some embodiments of any of the aspects described herein, a GEM cell cycle-dependent nuclear destruction tag can comprise a sequence of SEQ ID NO: 6 or amino acid residues 1-110, 1-60, 1-209, and 20-110 of the polypeptide encoded by the nucleic acid sequence of SEQ ID NO: 5 In some embodiments of any of the aspects described herein, a GEM cell cycle-dependent nuclear destruction tag can consist of a sequence of SEQ ID NO: 6 or amino acid residues 1-110, 1-60, 1-209, and 20-110 of the polypeptide encoded by the nucleic acid sequence of SEQ ID NO: 5.

TABLE 11

| | | SEQ |
|---|---|---|
| | | ID NO: |
| CDT1 | cccgcctctt cctcccttcc ttctttcctt gctttcgccg cgcactccgc cgccatggag | 3 |
| mRNA | cagcgccgcg tcaccgactt cttcgcgcgc cgccgccccg ggcccccccg catcgcgccg | |
| sequence | cccaagctgg cctgccgcac ccccagcccc gccaggcccg cactccgcgc cccggcctcc | |
| (NCBI | gctaccagtg gcagccgcaa gcgcgcccgc ccgcccgccg cccccggacg cgaccaggcc | |
| Ref Seq: | aggccaccgg cccgcaggag actgcggctg tcggtggacg aggtttccag ccccagtacc | |
| NM_030928.3) | cccgaggccc cagacatccc agcctgccct tctccgggcc agaagataaa gaaatccacc | |
| | ccggcagcag gtcagccgcc ccacctgaca tccgcgcagg accaggacac catctctgag | |
| | cttgcgtcat gcctgcaacg ggcccgggag ctgggggcaa gagtccgggc gctgaaggcc | |
| | agtgcccagg atgctgggga gtcctgcacc ccagaggccg agggccgccc tgaggagcca | |
| | tgtggcgaga aggcgcccgc ctaccagcgc ttccatgccc tggcccagcc cggcctgccg | |
| | ggactcgtgc tgccctacaa gtaccaggtg ctggcggaga tgttccgcag catggacacc | |
| | atcgtgggca tgctccacaa ccgctccgag acgcccacct ttgccaaggt ccagcggggc | |
| | gtccaggaca tgatgcgtag gcgttttgag gagtgcaatg ttggccagat caaaaccgtg | |
| | tacccggcct cctaccgctt ccgccaggag cgcagtgtcc ccaccttcaa ggatggcacc | |
| | aggaggtcag attaccagct caccatcgag ccactgctgg agcaggaggc tgacggagca | |
| | gccccccagc tcacggcctc gcgcctcctg cagcgacggc agatcttcag ccagaagctg | |
| | gtggagcatg tcaaggagca ccacaaggcc ttcctggcct ccctgagccc cgccatggtg | |
| | gtgccggagg accagctgac ccgctggcac ccgcgcttca acgtggatga agtacccgac | |
| | atcgagccgg ccgcgctgcc ccagccaccc gccacggaga agctcaccac tgctcaggag | |
| | gtgctggccc gggcccgcaa cctgatttca cccaggatgg agaaggcctt gagtcaattg | |
| | gccctgcgct ctgctgcgcc cagcagcccc gggtctccca ggccagcact gccggctacc | |
| | ccaccagcca ccccgcctgc agcctctccc agtgctctga agggggtgtc ccaggatctg | |
| | ctggagcgga tccgagccaa ggaggcacag aagcagctgg cacagatgac gcggtgcccg | |
| | gagcaggagc agcggctgca gcgcttagaa cggctgcctg agctggcccg cgtgctgcgg | |
| | agcgtctttg tgtccgaacg caagcctgcg ctcagcatgg aggtggcctg tgccaggatg | |
| | gtgggcagct gttgtactat catgagccct ggggaaatgg agaagcacct gctgctcctc | |
| | tccgagctgc tgccggactg gctcagcctc caccgcatcc gcaccgacac ctacgtcaag | |
| | ctggacaagg ccgcggacct cgcccacatc actgcacgcc tggcccacca gacacgtgct | |
| | gaggaggggc tgtgagcctg ggggccactg tggacagacg tgggcttcag aagctcgctg | |
| | gcctgggccc accagcattt tcttttatga acatgataca ctttggcctt cctttcccca | |
| | gcgcccctga gggccagagg cagatgtggg ctgcaggctg cacagcccga gggtctctgg | |
| | ctgcgggcgg tgggcccctt catggggctc acctggtgga ttcacattaa accggtttct | |
| | gtgggcacct ctgtccttgc tgctggtggg gaagggaagc cagatccagc accccctggg | |
| | gggccatcgg gagtgtggct gggggtgaag ggggctctgt ggcaatatgg ggttgggtag | |
| | tgtgggtggc aggccatccc ctctaatctt ggaacctctg aatatgggac ctcccacagc | |
| | aaagggtgac ttttgtcatt aagaaagact ggggtgggtg tggtggctca cgcctgtaac | |
| | cccagcactt tgggaggcca aggtgggcag atcacgaggt caagagatcg agaccatcct | |
| | ggcgaacatg gtgaaacccc atctctacta aaaatacaaa aaattagccg ggtgtggtgg | |
| | tgggcacctg tcgtcccagc tactagggag gctgaggcag gagaatggtg tgaacccagg | |
| | aggcacagct tgcagtgagc gaagatcgca ccactgcacg cactccagcc tgggtgacag | |
| | agcgagactc cgtctcaaaa aaaaaaattt caagactgga gaggtgatcc tgaattgtcc | |
| | agctacgccc catgtcatca cagggccttc atgacagggc cagagccagc cagctttgaa | |
| | gacgcggccc tgccccgaca caggcagcct ggagaagctg ggcaggacaa gtaggacatc | |
| | cctggagcct ccagaaggga ctggcctctg cccacacctt gacttcagta tttctgacct | |
| | cctaaactct aataaagtca tgcttacagc cactaaaaaa aaaaaaaaaa aaaaaaaaaa | |
| | aaaaaaaaaa aaaaaaaaaa aaaaaaaaaa aaaaaaaaaa aa | |
| CDT1 | PSPARPALRAPASATSGSRKRARPPAAPGRDQARPPARRRLRLSVDEVSSPSTPEAPDIPACPSP | 4 |
| tag 30-120 | GQKIKISTPAAGQPPHLTSAQDQDTI | |
| (amino acid | | |
| residues | | |
| 30-120 of | | |
| polypeptide | | |
| encoded by | | |
| DNA | | |
| (SEQ ID NO: 3) | | |
| GEM | gtctgcgtca gttggtcacg tggttgttcg gagcgggcga gcggagttag cagggcttta | 5 |
| mRNA sequence | ctgcagagcg cgccgggcac tccagcgacc gtggggatca gcgtaggtga gctgtggcct | |
| (GMNN) (NC | tttgcgaggt gctgcagcca tagctacgtg cgttcgctac gaggattgag cgtctccacc | |
| BI Ref Seq: | cagtaagtgg gcaagaggcg gcaggaagtg ggtacgcagg ggcgcaaggc gcacagcctc | |
| NM_015895.4) | tagacgactc gctttccctc cggccaacct ctgaagccgc gtcctacttt gacagctgca | |
| | gggccgcggc ctggtcttct gtgcttcacc atctacataa tgaatcccag tatgaagcag | |
| | aaacaagaag aaatcaaaga gaatataaag aatagttctg tcccaagaag aactctgaag | |
| | atgattcagc cttctgcatc tggatctctt gttggaagag aaaatgagct gtccgcaggc | |
| | ttgtccaaaa ggaaacatcg gaatgaccac ttaacatcta caacttccag ccctggggtt | |
| | attgtcccag aatctagtga aaataaaaat cttggaggag tcacccagga gtcatttgat | |
| | cttatgatta aagaaaatcc atcctctcag tattggaagg aagtggcaga aaaacggaga | |
| | aaggcgctgt atgaagcact taaggaaaat gagaaacttc ataaagaaat tgaacaaaag | |
| | gacaatgaaa ttgcccgcct gaaaaaggag aataaagaac tggcagaagt agcagaacat | |
| | gtacagtata tggcagagct aatagagaga ctgaatggtg aacctctgga taattttgaa | |
| | tcactggata atcaggaatt tgattctgaa gaagaaactg ttgaggattc tctagtggaa | |
| | gactcagaaa ttggcacgtg tgctgaagga actgtatctt cctctacgga tgcaaagcca | |
| | tgtatatgaa atgcattaat atttgactgt tgagaatttt actgccgaag tttacctcca | |
| | ctagttcttt gtagcagagt acataactac ataatgccaa ctctggaatc aaatttcctt | |
| | gtttgaatcc tgggacccta ttgcattaaa gtacaaatac tatgtatttt taatctatga | |
| | tggtttatgt gaataggatt ttctcagttg tcagccatga cttatgttta ttactaaata | |
| | aacttcaaac tcctgttgaa cattgtgtat aacttagaat aatgaaatat aaggagtatg | |
| | tgtagaaaaa aaaaa | |
| GEM | MNPSMKQKQEEIKENIKNSSVPRRTLKMIQPSASGSLVGRENELSAGLSKRKHRNDHLTSTTSSP | 6 |
| tag 1-110 | GVIVPESSENKNLGGVTQESFDLMIKENPSSQYWKEVAEKRRKAL | |
| (amino acid | | |
| residues 1-110 | | |
| of the | | |
| polypeptide | | |
| encoded by | | |
| SEQ ID NO: 5) | | |
| CDC6 | gagcgcggct ggagtttgct gctgccgctg tgcagtttgt tcaggggctt gtggtggtga | 7 |
| mRNA sequence | gtccgagagg ctgcgtgtga gagacgtgag aaggatcctg cactgaggag gtggaaagaa | |
| (NCBI Ref Seq: | gaggattgct cgaggaggcc tggggtctgt gaggcagcgg agctgggtga aggctgcggg | |
| NM_001254.3) | ttccggcgag gcctgagctg tgctgtcgtc atgcctcaaa cccgatccca ggcacaggct | |
| | acaatcagtt ttccaaaaag gaagctgtct cgggcattga acaaagctaa aaactccagt | |
| | gatgccaaac tagaaccaac aaatgtccaa accgtaacct gttctcctcg tgtaaaagcc | |
| | ctgcctctca gccccaggaa acgtctgggc gatgacaacc tatgcaacac tccccattta | |
| | cctccttgtt ctccaccaaa gcaaggcaag aaagagaatg gtccccctca ctcacataca | |
| | cttaagggac gaagattggt atttgacaat cagctgacaa ttaagtctcc tagcaaaaga | |
| | gaactagcca aagttcacca aaacaaaata ctttcttcag ttagaaaaag tcaagagatc | |
| | acaacaaatt ctgagcagag atgtccactg aagaaagaat ctgcatgtgt gagactattc | |
| | aagcaagaag gcacttgcta ccagcaagca aagctggtcc tgaacacagc tgtcccagat | |
| | cggctgcctg ccagggaaag ggagatggat gtcatcagga atttcttgag ggaacacatc | |
| | tgtgggaaaa aagctggaag cctttacctt tctggtgctc ctggaactgg aaaaactgcc | |
| | tgcttaagcc ggattctgca agacctcaag aaggaactga aaggctttaa aactatcatg | |
| | ctgaattgca tgtccttgag gactgcccag gctgtattcc cagctattgc tcaggagatt | |
| | tgtcaggaag aggtatccag gccagctggg aaggacatga tgaggaaatt ggaaaaacat | |
| | atgactgcag agaagggccc catgattgtg ttggtattgg acgagatgga tcaactggac | |
| | agcaaaggcc aggatgtatt gtacacgcta tttgaatggc catggctaag caattctcac | |
| | ttggtgctga ttggtattgc taataccctg gatctcacag atagaattct acctaggctt | |
| | caagctagag aaaaatgtaa gccacagctg ttgaacttcc caccttatac cagaaatcag | |
| | atagtcacta ttttgcaaga tcgacttaat caggtatcta gagatcaggt tctggacaat | |
| | gctgcagttc aattctgtgc ccgcaaagtc tctgctgttt caggagatgt tcgcaaagca | |
| | ctggatgttt gcaggagagc tattgaaatt gtagagtcag atgtcaaaag ccagactatt | |
| | ctcaaaccac tgtctgaatg taaatcacct tctgagcctc tgattcccaa gagggttggt | |
| | cttattcaca tatcccaagt catctcagaa gttgatggta acaggatgac cttgagccaa | |
| | gaaggagcac aagattcctt ccctcttcag cagaagatct tggtttgctc tttgatgctc | |
| | ttgatcaggc agttgaaaat caaagaggtc actctgggga agttatatga agcctacagt | |
| | aaagtctgtc gcaaacagca ggtggcggct gtggaccagt cagagtgttt gtcactttca | |
| | gggctcttgg aagccagggg cattttagga ttaaagagaa acaaggaaac ccgtttgaca | |
| | aaggtgtttt tcaagattga agagaaagaa atagaacatg ctctgaaaga taaagcttta | |
| | attggaaata tcttagctac tggattgcct taaattcttc tcttacaccc cacccgaaag | |
| | tattcagctg gcatttagag agctacagtc ttcattttag tgctttacac attcgggcct | |
| | gaaaacaaat atgacctttt ttacttgaag ccaatgaatt ttaatctata gattctttaa | |
| | tattagcaca gaataatatc tttgggtctt actattttta cccataaaag tgaccaggta | |
| | gacccttttt aattacattc actacttcta ccacttgtgt atctctagcc aatgtgcttg | |
| | caagtgtaca gatctgtgta gaggaatgtg tgtatattta cctcttcgtt tgctcaaaca | |
| | tgagtgggta tttttttgtt tgtttttttt gttgttgttg tttttgaggc gcgtctcacc | |
| | ctgttgccca ggctggagtg caatggcgcg ttctctgctc actacagcac ccgcttccca | |
| | ggttgaagtg attctcttgc ctcagcctcc cgagtagctg ggattacagg tgcccaccac | |
| | cgcgcccagc taatttttta atttttagta gagacagggt tttaccatgt tggccaggct | |
| | ggtcttgaac tcctgaccct caagtgatct gcccaccttg gcctccctaa gtgctgggat | |
| | tataggcgtg agccaccatg ctcagccatt aaggtatttt gttaagaact ttaagtttag | |
| | ggtaagaaga atgaaaatga tccagaaaaa tgcaagcaag tccacatgga gatttggagg | |
| | acactggtta aagaatttat ttctttgtat agtatactat gttcatggtg cagatactac | |
| | aacattgtgg cattttagac tcgttgagtt tcttgggcac tcccaagggc gttggggtca | |
| | taaggagact ataactctac agattgtgaa tatatttatt ttcaagttgc attctttgtc | |
| | tttttaagca atcagatttc aagagagctc aagctttcag aagtcaatgt gaaaattcct | |
| | tcctaggctg tcccacagtc tttgctgccc ttagatgaag ccacttgttt caagatgact | |
| | actttggggt tgggttttca tctaaacaca tttttccagt cttattagat aaattagtcc | |
| | atatggttgg ttaatcaaga gccttctggg tttggtttgg tggcattaaa tgg | |
| | ggatcctg | 8 |

An additional exemplary G1-restricting cell cycle-dependent nuclear destruction tag is a polypeptide sequence encoded by a nucleic acid sequence corresponding to nucleotides 93-100 of CDC6, e.g., nucleotides 93-100 of SEQ ID NO: 7, e.g., SEQ ID NO: 8.

An additional exemplary cell cycle-dependent nuclear destruction tag is a sequence encoded by a nucleic acid sequence corresponding to residues 3-14 of CDT1, e.g.,

SEQ ID NO: 9)
QRRVTDFFARRR.

An additional exemplary G1-restricting cell cycle-dependent nuclear destruction tag is a sequence QTPKRNPPLQKPPMKSLHKK (SEQ ID NO: 10) of RAG2. See, e.g, Li et al Immunity 5:575; which is incorporated by reference herein in its entirety.

In some embodiment of any of the aspects, the G1-restricting cell cycle-dependent nuclear destruction tag can comprise the sequence RXXLXXXXN (SEQ ID NO: 11) or KENXXXN (SEQ ID NO: 12). Further discussion can found, e.g, at Heo, J., Eki, R., and Abbas, T. (2016). Semin Cancer Biol 36, 33-51; Jiang, H., Chang, F. C., Ross, A. E., Lee, J., Nakayama, K., Nakayama, K., and Desiderio, S. (2005). Mol Cell 18, 699-709; and Teixeira, L. K., and Reed, S. I. (2013). Annu Rev Biochem 82, 387-414; each of which is incorporated by reference herein in its entirety.

The proteins and cell cycle-dependent nuclear destruction tags described herein can be obtained from any source, e.g., they can originate in humans, primates, rats, mice, rabbits and/or other mammals; or lower organisms, including frogs, flies and worms; or derived from the yeast S. cerevisiae or other unicellular organisms that are used to support genome engineering or protein expression. In some embodiments of any of the aspects described herein, the cell cycle-dependent nuclear destruction tag can comprise a sequence obtained from a protein having the same species of origin as the cell in which the user intends to use the engineered endonuclease, e.g., if the user intends to use the engineered endonuclease in a human cell, the cell cycle-dependent nuclear destruction tag can be human in origin.

In some embodiments of any of the aspects described herein, the cell cycle-dependent nuclear destruction tag can be located C-terminal of the endonuclease polypeptide. In some embodiments of any of the aspects described herein, the cell cycle-dependent nuclear destruction tag can be located N-terminal of the endonuclease polypeptide. In some embodiments of any of the aspects described herein, an engineered endonuclease can comprise two or more cell cycle-dependent nuclear destruction tags and/or two or more copies of any particular cell cycle-dependent nuclear destruction tag.

In some embodiments of any of the aspects described herein, a linker sequence can be provided between the cell cycle-dependent nuclear destruction tag and the endonuclease polypeptide. In some embodiments of any of the aspects described herein, an engineered endonuclease can comprise, from N-terminal to C-terminal, 1) an endonuclease polypeptide, 2) a linker sequence, and 3) a cell cycle-dependent nuclear destruction tag. In some embodiments of any of the aspects described herein, an engineered endonuclease can consist of, from N-terminal to C-terminal, 1) an endonuclease polypeptide, 2) a linker sequence, and 3) a cell cycle-dependent nuclear destruction tag. In some embodiments of any of the aspects described herein, an engineered endonuclease can consist of, essentially, from N-terminal to C-terminal, 1) an endonuclease polypeptide, 2) a linker sequence, and 3) a cell cycle-dependent nuclear destruction tag.

In some embodiments of any of the aspects described herein, a linker sequence can be provided between the cell cycle-dependent nuclear destruction tag and the endonuclease polypeptide. In some embodiments of any of the aspects described herein, an engineered endonuclease can comprise, from N-terminal to C-terminal, 1) an endonuclease polypeptide, 2) a linker sequence, and 3) at least one a cell cycle-dependent nuclear destruction tag. In some embodiments of any of the aspects described herein, an engineered endonuclease can consist of, from N-terminal to C-terminal, 1) an endonuclease polypeptide, 2) a linker sequence, and 3) at least one cell cycle-dependent nuclear destruction tag. In some embodiments of any of the aspects described herein, an engineered endonuclease can consist of, essentially, from N-terminal to C-terminal, 1) an endonuclease polypeptide, 2) a linker sequence, and 3) at least one cell cycle-dependent nuclear destruction tag.

As used herein, “linker” refers to refers to an amino acid sequence that serves the structural purpose of separating two other sequences in the same peptide chain. Linker design, selection, and exemplary linkers are well-known in the art and described, e.g., in Chen, X., et al, “Fusion protein linkers: proterty, design and functionality” Adv. Drug Deliv. Rev. (2013); which is incorporated by reference herein in its entirety.

In some embodiments of any of the aspects described herein, the linker sequence can be a flexible peptide sequence. In some embodiments of any of the aspects described herein, a linker can comprise glycine and serine residues. In some embodiments of any of the aspects described herein, a linker can consist essentially of glycine and serine residues. In some embodiments of any of the aspects described herein, a linker can consist of glycine and serine residues.

In some embodiments of any of the aspects described herein, the linker sequence can comprise the sequence GGGGS (SEQ ID NO: 2). In some embodiments of any of the aspects described herein, the linker sequence can consist of the sequence GGGGS (SEQ ID NO: 2). In some embodiments of any of the aspects described herein, the linker sequence can consist essentially of the sequence GGGGS (SEQ ID NO: 2).

In one aspect of any of the embodiments, described herein are Cas9^{D10A} (nicks) and Cas9 (DSBs) expression constructs that carry tags derived from the CDT1 and GEM (GEM) cell cycle regulators, which specify degradation of the fused protein outside G1 or S-G2/M phases of the cell cycle, respectively.

In one aspect of any of the embodiments, provided herein is an isolated nucleic acid molecule encoding an engineered endonuclease as described herein. In one aspect of any of the embodiments, provided herein is an isolated nucleic acid molecule capable of expressing an engineered endonuclease as described herein. In some embodiments of any of the aspects described herein, the sequence encoding the engineered endonuclease can be operably linked to a promoter.

In one aspect of any of the embodiments, provided herein is a vector comprising a nucleic acid encoding an engineered endonuclease as described herein. In some embodiments of any of the aspects described herein, a nucleic acid encoding an engineered endonuclease as described herein is comprised by a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences operably linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed from DNA to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

A nucleic acid molecule, such as DNA, is said to be capable of expressing a polypeptide if it contains nucleotide sequences which contain transcriptional and translational regulatory information and such sequences are “operably linked” to nucleotide sequences which encode the polypeptide. An operable linkage is a linkage in which the regulatory DNA sequences and the DNA sequence sought to be expressed are connected in such a way as to permit gene expression as peptides in recoverable amounts. The precise nature of the regulatory regions needed for gene expression may vary from organism to organism, as is well known in the analogous art.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

By “recombinant vector” is meant a vector that includes a heterologous nucleic acid sequence, or “transgene” that is capable of expression in vivo. It should be understood that the vectors described herein can, in some embodiments, be combined with other suitable compositions. In some embodiments of any of the aspects described herein, the vector is episomal. The use of a suitable episomal vector provides a means of maintaining the nucleotide of interest in the subject in high copy number extra chromosomal DNA thereby eliminating potential effects of chromosomal integration.

In one aspect of any of the embodiments, provided herein is a cell comprising a nucleic acid encoding an engineered endonuclease as described herein. In some embodiments of any of the aspects described herein, the nucleic acid encoding an engineered endonuclease can be stably integrated into the genome of the cell. In some embodiments of any of the aspects described herein, the nucleic acid encoding an engineered endonuclease can be constitutively transcriptionally active in the cell (e.g. operably linked to a constitutive promoter). In some embodiments of any of the aspects described herein, the nucleic acid encoding an engineered endonuclease can be inducibly transcriptionally active in the cell (e.g. operably linked to an inducible promoter). In some embodiments of any of the aspects described herein, a vector comprises the nucleic acid encoding an engineered endonuclease.

In one aspect of any of the embodiments, provided herein is a composition comprising 1) an engineered endonuclease as described herein; a nucleic acid molecule comprising a nucleic acid sequence encoding an engineered endonuclease as described herein; or a vector comprising a nucleic acid sequence encoding an engineered endonuclease as described herein; and 2) a donor nucleic acid.

When the action of an endonuclease results in the repair of the cleaved sequence (i.e., the target sequence or target nucleic acid molecule) via HDR, the cell will conduct the repair in a manner that utilizes a donor sequence. If a donor molecule is provided to the cell, it is possible for a specific desired alteration (the sequence of the alteration being comprised by the donor) to be made as a result of HDR. As used herein, “donor nucleic acid” refers to a nucleic acid molecule comprising a sequence that is to be copied or incorporated into a target nucleic acid molecule. The sequence to be incorporated can be introduced into the target nucleic acid molecule via homology directed repair at the target sequence, thereby causing an alteration of the target sequence from the original target sequence to the sequence comprised by the donor nucleic acid. Accordingly, the sequence comprised by the donor nucleic acid can be, relative to the target sequence, an insertion, a deletion, an indel, a point mutation, a repair of a mutation, etc. The donor nucleic acid can be, e.g., a single-stranded DNA molecule; a double-stranded DNA molecule; a DNA/RNA hybrid molecule; and a DNA/modRNA (modified RNA) hybrid molecule.

The donor nucleic acid, in addition to the sequence that is to be incorporated into the target nucleic acid molecule, can comprise one or more regions flanking the sequence that is to be incorporated into the target nucleic acid molecule. The flanking regions can comprise sequences with homology to the target sequence and/or sequences flanking the target sequence, i.e., in order to hybridize with the target nucleic acid near the target sequence and permit HDR to occur. Design of donor nucleic acids, particularly with respect to flanking region(s) is discussed in the art e.g., in Richardson et al., Nat. Biotech., 2016; or Davis and Maizels, Cell Reports, 2016, which are incorporated by reference herein in its entirety.

In one aspect of any of the embodiments, provided herein is a composition comprising 1) an engineered endonuclease as described herein wherein the endonuclease polypeptide comprises a Cas9 or Cas9-derived polypeptide; a nucleic acid molecule comprising a nucleic acid sequence encoding an engineered endonuclease as described herein wherein the endonuclease polypeptide comprises a Cas9 or Cas9-derived polypeptide; or a vector comprising a nucleic acid sequence encoding an engineered endonuclease as described herein wherein the endonuclease polypeptide comprises a Cas9 or Cas9-derived polypeptide; and 2) a donor nucleic acid sequence. In one aspect of any of the embodiments, provided herein is a composition comprising 1) an engineered endonuclease as described herein wherein the endonuclease polypeptide comprises a Cas9 or Cas9-derived polypeptide; a nucleic acid molecule comprising a nucleic acid sequence encoding an engineered endonuclease as described herein wherein the endonuclease polypeptide comprises a Cas9 or Cas9-derived polypeptide; or a vector comprising a nucleic acid sequence encoding an engineered endonuclease as described herein wherein the endonuclease polypeptide comprises a Cas9 or Cas9-derived polypeptide; 2) a donor nucleic acid sequence; and 3) one or more crRNA, tracrRNA, or sgRNA molecules. In one aspect of any of the embodiments, provided herein is a composition comprising 1) an engineered endonuclease as described herein wherein the endonuclease polypeptide comprises a Cas9 or Cas9-derived polypeptide; a nucleic acid molecule comprising a nucleic acid sequence encoding an engineered endonuclease as described herein wherein the endonuclease polypeptide comprises a Cas9 or Cas9-derived polypeptide; or a vector comprising a nucleic acid sequence encoding an engineered endonuclease as described herein wherein the endonuclease polypeptide comprises a Cas9 or Cas9-derived polypeptide; and 2) one or more crRNA, tracrRNA, or sgRNA molecules. Design of crRNA, tracrRNA, or sgRNA molecules and, e.g., production of CRISPR ribonucleoproteins is known in the art and described, for example in Anders et al. 2014 Methods in Enzymology 546:1-20; which is incorporated by reference herein in its entirety.

In one aspect of any of the embodiments, described herein is a method of modifying the sequence of a target nucleic acid molecule, the method comprising: contacting the target nucleic acid molecule with an engineered endonuclease as described herein. In some embodiments of any of the aspects described herein, the engineered endonuclease has been programmed and/or engineered to cleave the target nucleic acid at the locus selected for modification. In some embodiment of any of the aspects, a cell comprises the target nucleic acid molecule.

In one aspect of any of the embodiments, described herein is a method of modifying the sequence of a target nucleic acid molecule via homologous recombination (HR), the method comprising: contacting the target nucleic acid molecule with an engineered endonuclease comprising an endonuclease polypeptide and a G1-restricting cell cycle-dependent nuclear destruction tag. In some embodiments of any of the aspects described herein, the G1-restricting cell cycle-dependent nuclear destruction tag can be a CDT1 cell cycle-dependent nuclear destruction tag.

Non-homologous end joining (NHEJ) is a process by which double-stranded breaks in DNA are repaired. Two ends generated by one or more DSBs are ligated together and since a donor is not used, the repair typically generates changes in the sequence relative to the sequence that existed prior to the DSB's formation. NHEJ is noted for a high rate of mutation and when donors are incorporated at the targeted locus, the incorporation has a low level of precision. In one aspect of any of the embodiments, provided herein is a method of modifying the sequence of a target nucleic acid molecule via non-homologous end-joining (NHEJ) and/or mutEJ, the method comprising: contacting the target nucleic acid molecule with an engineered endonuclease comprising an endonuclease polypeptide and a S-G2/M-restricting cell cycle-dependent nuclear destruction tag. In some embodiments of any of the aspects described herein, the S-G2/M-restricting cell cycle-dependent nuclear destruction tag is a GEM cell cycle-dependent nuclear destruction tag

In some embodiments of any of the aspects described herein, a target nucleic acid molecule can be further contacted with a donor nucleic acid sequence. In some embodiments of any of the aspects described herein, the donor nucleic acid sequence is provided separately from the engineered endonuclease. In some embodiments of any of the aspects described herein, the donor nucleic acid sequence is provided concurrently with the engineered endonuclease, e.g., in the same composition or encoded in the same vector.

In some embodiments of any of the aspects described herein, contacting the target nucleic acid molecule with an engineered endonuclease as described herein can comprise contacting a cell comprising the target nucleic acid molecule with the engineered endonuclease. For example, the engineered endonuclease can be delivered to the cell as a protein and/or RNP; the cell can be contacted with a vector encoding the engineered endonuclease; or the cell can have a stably integrated nucleic acid molecule encoding the engineered endonuclease.

In some embodiments of any of the aspects described herein, wherein the engineered endonuclease comprises a Cas9 or Cas9-derived endonuclease polypeptide and the method can further comprise contacting the target nucleic acid molecule with one or more crRNA, tracrRNA, or sgRNA molecules. In some embodiments of any of the aspects described herein, the engineered endonuclease and one or more crRNA, tracrRNA, or sgRNA molecules can be provided as an RNP. In some embodiments of any of the aspects described herein, the engineered endonuclease and one or more crRNA, tracrRNA, or sgRNA molecules can be provided as a polypeptide and a nucleic acid molecule, separately or in the same composition. In some embodiments of any of the aspects described herein, one or both of the engineered endonuclease and one or more crRNA, tracrRNA, or sgRNA molecules can be provided as one or more vectors encoding the engineered endonuclease and one or more crRNA, tracrRNA, or sgRNA molecules. It is contemplated herein that the engineered endonuclease and one or more crRNA, tracrRNA, or sgRNA molecules can be provided in any combination of the foregoing forms.

In one aspect, described herein is a kit comprising a composition as described herein, e.g., an engineered endonuclease, a vector comprising a nucleic acid sequencing encoding an engineered endonuclease, a cell comprising an engineered endonuclease or a nucleic acid encoding an engineered endonuclease; or a nucleic acid encoding an engineered endonuclease. A kit is any manufacture (e.g., a package or container) comprising at least one reagent, e.g., an engineered endonuclease, the manufacture being promoted, distributed, or sold as a unit for performing the methods described herein.

The kits described herein can optionally comprise additional components useful for performing the methods described herein. By way of example, the kit can comprise fluids (e.g., buffers) suitable for a composition comprising an engineered endonuclease as described herein, an instructional material which describes performance of a method as described herein, donor nucleic acid molecules, sgRNA, crRNA, and/or tracrRNA and the like. A kit can further comprise devices and/or reagents for delivery of the composition as described herein. Additionally, the kit may comprise an instruction leaflet and/or may provide information as to the relevance of the obtained results.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

In some embodiments, the endonuclease can be an engineered endonuclease. As used herein, “engineered” refers to the aspect of having been manipulated by the hand of man. For example, a nuclease is considered to be “engineered” when the sequence of the nuclease is manipulated by the hand of man to differ from the sequence of the nuclease as it exists in nature. As is common practice and is understood by those in the art, progeny and copies of an engineered polynucleotide and/or polypeptide are typically still referred to as “engineered” even though the actual manipulation was performed on a prior entity.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. endonuclease activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA, crRNA, tracrRNA, sgRNA and the like.

In some embodiments of any of the aspects described herein, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments of any of the aspects described herein, a composition as described herein can be a pharmaceutical composition. As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects described herein, a pharmaceutically acceptable carrier can be a carrier other than water. In some embodiments of any of the aspects described herein, a pharmaceutically acceptable carrier can be a cream, emulsion, gel, liposome, nanoparticle, and/or ointment. In some embodiments of any of the aspects described herein, a pharmaceutically acceptable carrier can be an artificial or engineered carrier, e.g., a carrier that the active ingredient would not be found to occur in in nature.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

In some embodiments of any of the aspects described herein, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:

1. A nucleic acid construct comprising:
(a) a first nucleotide sequence that expresses an endonuclease, operably linked to
(b) a second nucleotide sequence that expresses either a region of Geminin (GEM) polypeptide or a region of chromatin licensing and DNA replication factor 1 (CDT1) polypeptide or another polypeptide targeted for cell cycle-dependent nuclear destruction, wherein the second nucleotide sequence is operably linked to the first nucleotide sequence.
   2. The nucleic acid construct of paragraph 1, wherein the endonuclease is selected from the group consisting of Cas9^{D10A} or Cas9.
   3. A method of modifying the sequence of a target nucleic acid molecule, the method comprising contacting the target nucleic acid molecule with
   a) a donor nucleic acid molecule comprising the modification to be made in the target nucleic acid molecule;
   b) a first nucleotide sequence encoding a nickase selected from the group consisting of a nuclease with one active site disabled; I-Anil with one active site disabled; Cas9^{D10A}; or Cas9;
   c) a second nucleotide sequence that expresses either a region of Geminin (GEM) polypeptide or a region of chromatin licensing and DNA replication factor 1 (CDT1) polypeptide or another polypeptide targeted for cell cycle-dependent nuclear destruction, wherein the second nucleotide sequence is operably linked to the first nucleotide sequence.
   4. A method of modifying the sequence of a target nucleic acid molecule, the method comprising contacting the target nucleic acid molecule with
   a) a ssDNA donor nucleic acid molecule comprising the modification to be made in the target nucleic acid molecule;
   b) a first nucleotide sequence encoding a nuclease selected from the group consisting of nucleases comprising a FokI cleavage domain; zinc finger nucleases; TALE nucleases; RNA guided engineered nucleases; Cas9; Cas9-derived nucleases; Cfp1; Cfp1-derived nucleases; homing endonucleases; and other endonucleases that make targeted DNA nicks or double strand breaks; and
   c) a second nucleotide sequence that expresses either a region of the Geminin (GEM) polypeptide or a region of the chromatin licensing and DNA replication factor 1 (CDT1) polypeptide or another polypeptide targeted for cell cycle-dependent nuclear destruction, wherein the second nucleotide sequence is operably linked to the first nucleotide sequence.
   4. A method of restricting nuclear activity of CRISPR/Cas9 or CRISPR/Cas9^{D1} that modifies nucleic acids to G1 or to S-G2/M phase of the cell cycle in a host cell, the method comprising transfecting a host cell with a fusion construct comprising a nucleotide sequence that expresses the CRISPR/Cas9 or CRISPR/Cas9^{D10A} fused to a nucleotide sequence that expresses CDT1 or geminin (GEM), wherein a fusion construct expressing CDT1 restricts expression of the CRISPR/Cas9 or CRISPR/Cas9^{D10A} to G1 and a fusion construct expressing GEM restricts expression of the CRISPR/Cas9 or CRISPR/Cas9^{D10A} to S phase.

Some embodiments of the technology described herein can be defined according to any of the following numbered paragraphs:
1. An engineered endonuclease comprising an endonuclease polypeptide and a cell cycle-dependent nuclear destruction tag.
2. The engineered endonuclease of paragraph 1, wherein the endonuclease polypeptide comprises a sequence-specific endonuclease.
3. The engineered endonuclease of any of paragraphs 1-2, wherein the endonuclease polypeptide comprises an endonuclease selected from the group consisting of:
   Cas9; a Cas9-derived nuclease; Cas9^{D10A}; a Cas9 nickase variant; a TALEN; a ZFN; Cpf1; a nuclease comprising a FokI cleavage domain; a RNA-guided engineered nuclease; and a homing endonuclease.
4. The engineered endonuclease of any of paragraphs 1-3, wherein the cell cycle-dependent nuclear destruction tag comprises a sequence found in a protein selected from the group consisting of:
   GEM; CDT1; Orc1; Cdc25A; Cyclin A; Cyclin B1; Securin; Plk1; Cdc6; Cyclin E; c-Jun; c-Myc; and RAG-2.
5. The engineered endonuclease of any of paragraphs 1-4, wherein the cell cycle-dependent nuclear destruction tag comprises a Geminin (GEM) or chromatin licensing and DNA replication factor (CDT1) cell cycle-dependent nuclear destruction tag.
6. The engineered endonuclease of paragraph 4, wherein the cell cycle-dependent nuclear destruction tag is selected from SEQ ID NO: 4, SEQ ID NO: 6, or SEQ ID NOs: 8-12.
7. The engineered endonuclease of paragraph 4, wherein the cell cycle-dependent nuclear destruction tag comprises a sequence selected from SEQ ID NO: 4, SEQ ID NO: 6, or SEQ ID NOs: 8-12.
8. The engineered endonuclease of paragraph 4, wherein the cell cycle-dependent nuclear destruction tag corresponds to a sequence selected from SEQ ID NO: 4, SEQ ID NO: 6, or SEQ ID NOs: 8-12.
9. The engineered endonuclease of any of paragraphs 1-8, wherein the tag is located at the C-terminus of the endonuclease.
10. The engineered endonuclease of any of paragraphs 1-9, further comprising a linker sequence between the endonuclease polypeptide and cell cycle-dependent nuclear destruction tag.
11. The engineered endonuclease of paragraph 10, wherein the linker sequence comprises the sequence GGGGS (SEQ ID NO: 2).
12. An isolated nucleic acid molecule encoding the engineered endonuclease of any of paragraphs 1-11.
13. A vector comprising an isolated nucleic acid molecule encoding the engineered endonuclease of any of paragraphs 1-11.
14. A composition comprising:
   the engineered endonuclease of any of paragraphs 1-11; and
   a donor nucleic acid sequence.
15. The composition of paragraph 14, wherein the engineered endonuclease comprises a Cas9 or Cas9-derived endonuclease polypeptide and the composition further comprises one or more crRNA, tracrRNA, or sgRNA molecules.
16. A method of modifying the sequence of a target nucleic acid molecule, the method comprising:
   contacting the target nucleic acid molecule with the engineered endonuclease of any of paragraphs 1-11.
17. The method paragraph 16, wherein the target nucleic acid molecule is further contacted with a donor nucleic acid sequence.
18. The method of any of paragraphs 16-17, wherein the engineered endonuclease comprises a Cas9 or Cas9-derived endonuclease polypeptide and the method further comprises contacting the target nucleic acid molecule with one or more crRNA, tracrRNA, or sgRNA molecules.
19. The method of any of paragraphs 16-18, wherein the engineered endonuclease comprises an endonuclease polypeptide and a G1-restricting cell cycle-dependent nuclear destruction tag and the modification thereby occurs via homology-directed repair.
20. The method of paragraph 19, wherein the G1-restricting cell cycle-dependent nuclear destruction tag is a CDT1 cell cycle-dependent nuclear destruction tag.
21. The method of any of paragraphs 16-18, wherein the engineered endonuclease comprises an endonuclease polypeptide and a S-G2/M-restricting cell cycle-dependent nuclear destruction tag and the modification thereby occurs via non-homologous end-joining (NHEJ) or mutagenic end-joining (mutEJ).
22. The method of paragraph 21, wherein the S-G2/M-restricting cell cycle-dependent nuclear destruction tag is a GEM cell cycle-dependent nuclear destruction tag.

### EXAMPLES

### Example 1

Construction of Constructs Expressing Cas9 and Cas9^{D10A} carrying CDT1 and GEM Tags

To generate the pCDNA-Cas9-CDT1, pCDNA-Cas9-GEM, pCDNA-Cas9^{D10A}-CDT1 and pCDNA-Cas9^{D10A}-GEM expression constructs, the T2A-BFP tag in both pCDNACas9-T2A-BFP and pcDNA-Cas9^{D10A}-T2A-BFP [4] was replaced with the mKO2-hCDT1(30-120) and mAGhGEM(1-110) cell cycle tags [7], referred to here and previously [6] as the CDT1 and GEM tags.

Cloning was carried out as follows. First, the Mfe1 site in both pCDNA-Cas9-T2A-BFP and pCDNA-Cas9^{D10A}-T2A-BFP was destroyed by Mfe1 digestion, fill-in and religation. The plasmids were then digested with Not1 and Xba1, to remove the T2A-BFP cassette, which was replaced with a short duplex, LinkerMCS, which carries Not1-Mfe1-Hpa1-Nhe1 sites, and a linker encoding a pentapeptide (Gly-Gly-Gly-Gly-Ser) (SEQ ID NO: 2) between the Not1 and Mfe1 sites. Following digestion with Mfe1 and Nhe1, an EcoR1/Xba1 fragment from pCSII-EF-mKO2-hCDT1(30-120) [7] was cloned in to generate constructs tagged with CDT1:

pCas9^{D10A}-mKO2-CDT1

pCas9-mKO2-CDT1

To generate constructs tagged with GEM, these plasmids were digested with Nhe1, partially filled in using only dCTP and dTTP in the fill-in reaction, then digested with Mfe1 to remove the cassette bearing mKO2 and the CDT1 tag; and ligated to fragments carrying the GEM tag, generated by digestion of pCSII-EF-mAGhGEM(1-110) [7] with HinDIII, partial fill in in a reaction containing only dGTP and dATP, followed by digestion with EcoRI. This created four variants:

pCas9^{D10A}-mAG-GEM

pCas9-mAG-GEM

To remove the cassettes encoding the mAGS and mKO2 fluorescent proteins, plasmids were digested with Not1, overhangs filled in to maintain reading frame, and plasmids religated. This created four plasmids:

pCas9^{D10A}-CDT1

pCas9^{D10A}-GEM

pCas9-CDT1

pCas9-GEM

All constructs were verified by both restriction digestion and sequencing.

Cell Culture and Transfection

HEK 293T TL7 cells were seeded at a density of 7×10⁴ cells per well in a 24-well plate containing 500 μL of complete DMEM media (DMEM supplemented with 10% FBS, 10μ of 200 mM L-glutamine, 5 μL of 10,000 units/ml penicillin and 10 mg/ml streptomycin solution). The following day, the cells were transfected with 150 ng of Cas9 plasmid, 75 ng of gRNA, 150 ng of duplex plasmid donor pCS14GFP [4], or 0.4 μl of 33 μM single stranded oligonucleotide donor (SSO-2, sequence shown below), and 50 μL of the BRC3 dominant negative BRCA2 peptide [8] to activate alternative HDR, as indicated; and 1.2 Lipfofectamine LTX™ transfection reagent per transfection. The cells were then incubated at 37° C. and 5% CO₂ overnight. The cells were then washed once with 500 μL of Dulbecco's Phosphate Buffered Saline (DPBS) treated with 150 μL of 0.05% trypsin in DPBS and split into 6-well plates containing 2 ml of complete DMEM media as described above. The cells were incubated for two days at 37° C. and 5% CO₂. On the third day after transfection, the cells were washed with DPBS, harvested with 150 μL of 0.05% trypsin in DPBS and fixed in 150 μL of 4% paraformaldehyde. Data was collected using a BD Biosciences LSR II Flow Cytometer™

SS0-2:
(SEQ ID NO: 1)
5′-TGGACGGCGACGTAAACGGCCACAAGT TCAGCGTGTCCGGCgagg
gtgagggcgatgcCACCTACGGCAAGCTGACCCTGAAGTTCATCTGCAC
CACCG-3′

Uppercase letters denote arms of homology between SSO-2 and the target, and lowercase letters indicate the central region of heterology that must replace sequence in the target to generate a functional GFP gene.

Data Analysis

Each set of assays was performed in triplicate, and a mean frequency of both HDR and mutEJ was determined. The values presented represent the mean±SEM from a representative experiment. Two-tailed T-tests were performed to determine if the differences between HDR and mutEJ frequencies at different stages of cell cycle were statistically significant. These analyses were performed using Microsoft Excel™ (2015).

Control HDR and mutEJ frequencies were measured following transfection with a construct with pCDNA-Cas9^{D10A}-T2A-BFP that expressed Cas9^{D10A} endonuclease transcriptionally linked to BFP by a T2A linker, and with neither a CDT1 nor GEM tag.

Frequencies of cells expressing BFP provided a measure of transfection efficiency (33%) that enabled us to correct observed HDR and mutEJ frequencies in other experiments, by multiplying raw data by three. This correction was applied to experiments using tagged Cas9 or Cas9^{D10A} expressed from constructs that did not co-express BFP.

### RESULTS AND DISCUSSION

HDR and mutEJ frequencies were analyzed in HEK293T cells bearing the Traffic Light (TL) Reporter integrated at heterogeneous chromosomal sites. This reporter is designed so that HDR causes GFP expression and mutEJ enables mCherry expression, enabling efficiencies of both processes to be assayed as frequencies of GFP+ or mCherry+ cells by flow cytometry [1,3,4].

Frequencies of HDR and mutEJ initiated by DNA nicks or DSBs targeted to the TL reporter by Cas9 or Cas9^{D10A}, respectively, were measured using a 99 nt single-stranded oligonucleotide (SSO-2, complementary to the intact target strand) as donor for repair of nicks, and either a SSO or a duplex plasmid for repair of DSBs. We previously showed that HDR initiated by a nick using an SSO donor occurs most efficiently if canonical HDR is inhibited, as this activates an alternative HDR pathway that is very efficient at nicks. To do so cells were cultured with the BRC3 dominant negative peptide, which suppresses canonical HDR [8]. Results were expressed as HDR and mutEJ frequencies among all cells (Tables 1-4), and then corrected for transfection efficiency and expressed as frequencies among transfectants only (Tables 5-8).

This analysis demonstrated that DNA nicks initiate HDR much more efficiently in G1 phase than in S-G2/M phases (Tables 5, 6). Preferential HDR at nicks initiated in G1 phase is greatly stimulated in cells in which alternative HDR is stimulated by transfection with the BRC3 dominant negative peptide, which suppresses canonical HDR and activates the alternative HDR pathway (altHDR [4]). In that case, the frequency of HDR among transfectants is 21.2%; and the ratio of HDR to mutEJ is approximately 20:1.

This analysis further demonstrated that DNA DSBs initiate HDR more efficiently in S-G2/M phase than in G1 phase, using either a plasmid or SSO donor (Tables 7, 8). Preferential HDR of DSBs initiated in S phase is especially evident using a duplex DNA donor (7-fold) rather than an SSO donor (2.5-fold). Frequencies of HDR at DSBs are 3-fold higher using an SSO donor than a duplex plasmid donor; and mutEJ frequencies are unaffected by donor structure. A SSO donor supports more efficient HDR at DSBs than a plasmid donor, and generates HDR:mutEJ in a ratio slightly better than 2:1. This contrasts with the 20:1 HDR:mutEJ ratio for G1 phase initiated DNA nicks.

We conclude that initiating HDR with nicks in G1 phase offers a slightly more efficient and much safer approach to gene correction and engineering than initiating HDR with DSBs.

TABLE 1
DNA Nick Repair Frequencies Supported by an SSO Donor
(Among All Cells)

| | Cell Cycle | | |
|---|---|---|---|
| | Phase | HDR [symbol] (%) | [symbol] (%) |
| pCas9^{D10A}-CDT1 | G1 | 0.731 ± 0.13 | 0.123 ± 0.04 |
| pCas9^{D10A}-GEM | S-G2/M | 0.341 ± 0.10 | 0.106 ± 0.04 |
| pCas9^{D10A} | G1, S-G2/M | 0.240 ± 0.04 | 0.006 ± 0.01 |

TABLE 2
DNA Nick Repair Frequencies Supported by an SSO Donor
(Among All Cells, with Alternative HDR Stimulated by Expression
of BRC3)

| | Cell Cycle | | |
|---|---|---|---|
| | Phase | HDR [symbol] (%) | [symbol] (%) |
| pCas9^{D10A}-CDT1 | G1 | 7.07 ± 0.12 | 0.486 ± 0.08 |
| pCas9^{D10A}-GEM | S-G2/M | 1.54 ± 0.06 | 0.249 ± 0.01 |
| pCas9^{D10A} | G1, S-G2/M | 3.56 ± 0.20 | 0.163 ± 0.004 |

TABLE 3
DNA DSB Repair Frequencies Supported by a Plasmid Donor
(Among All Cells

| | Cell Cycle | | |
|---|---|---|---|
| | Phase | HDR [symbol] (%) | [symbol] (%) |
| pCas9-CDT1 | G1 | 0.21 ± 0.02 | 1.44 ± 0.03 |
| pCas9-GEM | S-G2/M | 1.4 ± 0.1 | 2.69 ± 0.11 |
| pCas9 | G1, S-G2/M | 1.14 ± 0.04 | 3.10 ± 0.11 |

TABLE 4
DNA DSB Repair Frequencies Supported by a SSO Donor
(Among All Cells)

| | Cell Cycle | | |
|---|---|---|---|
| | Phase | HDR (%) | [symbol] (%) |
| pCas9-CDT1 | G1 | 2.16 ± 0.24 | 1.93 ± 0.03 |
| pCas9-GEM | S-G2/M | 5.55 ± 0.14 | 2.44 ± 0.05 |
| pCas9 | G1, S-G2/M | 2.58 ± 0.06 | 1.74 ± 0.02 |

TABLE 5
DNA Nick Repair Frequencies Supported by a SSO Donor
(Among Transfected Cells)

| | Cell Cycle | | |
|---|---|---|---|
| | Phase | HDR (%) | [symbol] (%) |
| pCas9^{D10A}-CDT1 | G1 | 2.193 ± 0.40 | 0.370 ± 0.11 |
| pCas9^{D10A}-GEM | S-G2/M | 1.024 ± 0.29 | 0.318 ± 0.12 |
| pCas9^{D10A} | G1, S-G2/M | 0.720 ± 0.12 | 0.018 ± 0.02 |

TABLE 6
DNA Nick Repair Frequencies Supported by an SSO Donor
(Among Transfected Cells, with Alternative HDR Stimulated
by Expression of BRC3)

| | Cell Cycle | | |
|---|---|---|---|
| | Phase | HDR (%) | [symbol] (%) |
| pCas9^{D10A}-CDT1 | G1 | 21.20 ± 0.07 | 1.22 ± 0.64 |
| pCas9^{D10A}-GEM | S-G2/M | 4.63 ± 0.02 | 0.747 ± 0.26 |
| pCas9^{D10A} | G1, S-G2/M | 10.68 ± 0.14 | 0.490 ± 0.27 |

TABLE 7
DNA DSB Repair Frequencies Supported by a Plasmid Donor
(Among Transfected Cells)

| | Cell Cycle | | |
|---|---|---|---|
| | Phase | HDR (%) | [symbol] (%) |
| pCas9-CDT1 | G1 | 0.633 ± 0.06 | 4.33 ± 0.08 |
| pCas9-GEM | S-G2/M | 4.20 ± 0.02 | 8.06 ± 0.33 |
| pCas9 | G1, S-G2/M | 3.41 ± 0.11 | 9.29 ± 0.34 |

TABLE 8
DNA DSB Repair Frequencies Supported by a SSO Donor
(Among Transfected Cells)

| | Cell Cycle | | |
|---|---|---|---|
| | Phase | HDR [symbol] (%) | [symbol] (%) |
| pCas9-CDT1 | G1 | 6.47 ± 0.73 | 5.78 ± 0.10 |
| pCas9-GEM | S-G2/M | 16.66 ± 0.43 | 7.31 ± 0.14 |
| pCas9 | G1, S-G2/M | 7.75 ± 0.19 | 5.22 ± 0.06 |

### REFERENCES

1. Certo M T, Ryu B Y, Annis J E, Garibov M, Jarjour J, et al. (2011) Tracking genome engineering outcome at individual DNA breakpoints. Nat Methods 8: 671-676.
2. Cho S W, Kim S, Kim Y, Kweon J, Kim H S, et al (2014) Analysis of off-target effects of CRISPR/Cas-derived RNA-guided endonucleases and nickases. Genome Res 24: 132-141.
3. Davis L, Maizels N (2011) DNA nicks promote efficient and safe targeted gene correction. PLoS One 6: e23981.
4. Davis L, Maizels N (2014) Homology-directed repair of DNA nicks via pathways distinct from canonical double-strand break repair. Proc Natl Acad Sci USA 111: E924-932.
5. Karanam K, Kafri R, Loewer A, Lahav G (2012) Quantitative live cell imaging reveals a gradual shift between DNA repair mechanisms and a maximal use of H R in mid S phase. Mol Cell 47: 320-329.
6. Le Q, Maizels N (2015) Cell cycle regulates nuclear stability of AID and the cellular response to AID. PLoS Genetics 11:e1005411.
7. Sakaue-Sawano A, Kurokawa H, Morimura T, Hanyu A, Hama H, et al. (2008) Visualizing spatiotemporal dynamics of multicellular cell-cycle progression. Cell 132: 487-498.
8. Stark J M, Hu P, Pierce A J, Moynahan M E, Ellis N, et al (2002) ATP hydrolysis by mammalian RAD51 has a key role during homology-directed DNA repair. J Bioi Chem 277: 20185-20194.
9. Tsai S Q, Zheng Z, Nguyen N T, Liebers M, Topkar V V, et al. (2015) GUIDE-seq enables genome-wide profiling of off-target cleavage by CRISPR-Cas nucleases. Nat Biotechnol 33: 187-197.
10. Davis L, Maizels N (2016) Two distinct pathways support homology-directed repair at DNA nicks. Cell Reports 17:1872-1871.

### Example 2

Understanding nick repair can permit minimization of unwanted products of HDR and maximize the efficiency of gene therapy. Nicks are the most common form of DNA damage and can be repaired using single strand annealing or homology directed repair (HDR). Mutagenic end joining (mutEJ) is highly mutagenic but occurs infrequently at nicks. Nicks can be very efficiently be repaired by alternative HDR when canonical HDR is suppressed. Canonical HDR is thought to be most active in S phase following DNA replication. Certain types of solid tumors, such as ovarian and breast, are deficient in canonical HDR.

Described herein is the development of a Cas9-cell cycle tag fusion protein using cloning techniques and the determination of the efficacy of cell cycle presence and degradation of cell cycle tagged Cas9. HEK 293T TL7 cells were transfected with both cell cycle tagged Cas9WT (DSBs) and Cas9^{D10A} (nicks) along with a BFP tagged control Cas9 to measure HDR and NHEJ levels. HEK 293T TL7 cells were transfected with BRCA2 knockdown and both Cas9WT and Cas9^{D10A} along with an BFP tagged control Cas9 to measure HDR and NHEJ levels.

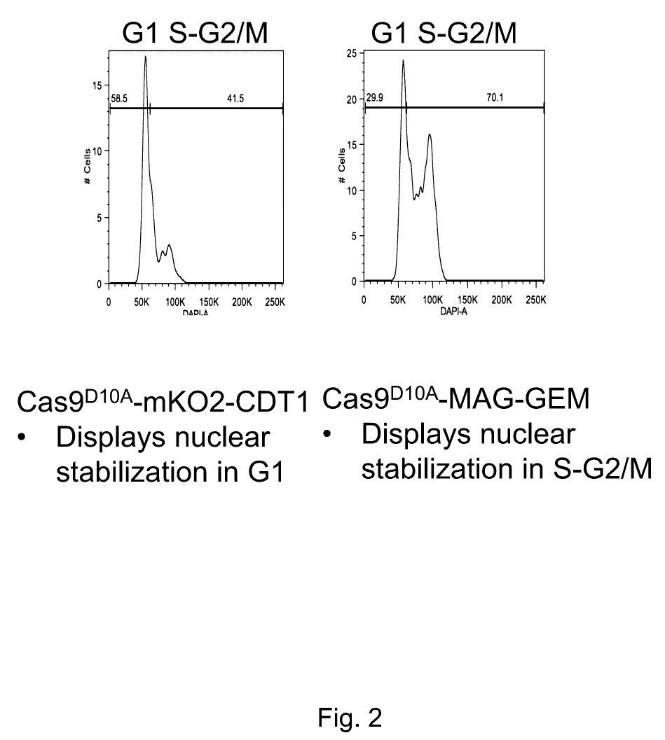

The tags imparted cell cycle-controlled protein stability as shown in Table 9 and FIG. 2.

TABLE 9

Nuclear
protein stable in:

| Cas9 Variant | Targeted Break | G1 | S-G2/M |
|---|---|---|---|
| Cas9^{D10A}-CDT1 | Nick | ✓ | — |
| Cas9^{D10A}-GEM | Nick | — | ✓ |
| Cas9^{D10A}-BFP | Nick | ✓ | ✓ |
| Cas9^{WT}-CDT1 | DSB | ✓ | — |
| Cas9^{WT}-GEM | DSB | — | ✓ |
| Cas9^{WT}-BFP | DSB | ✓ | ✓ |

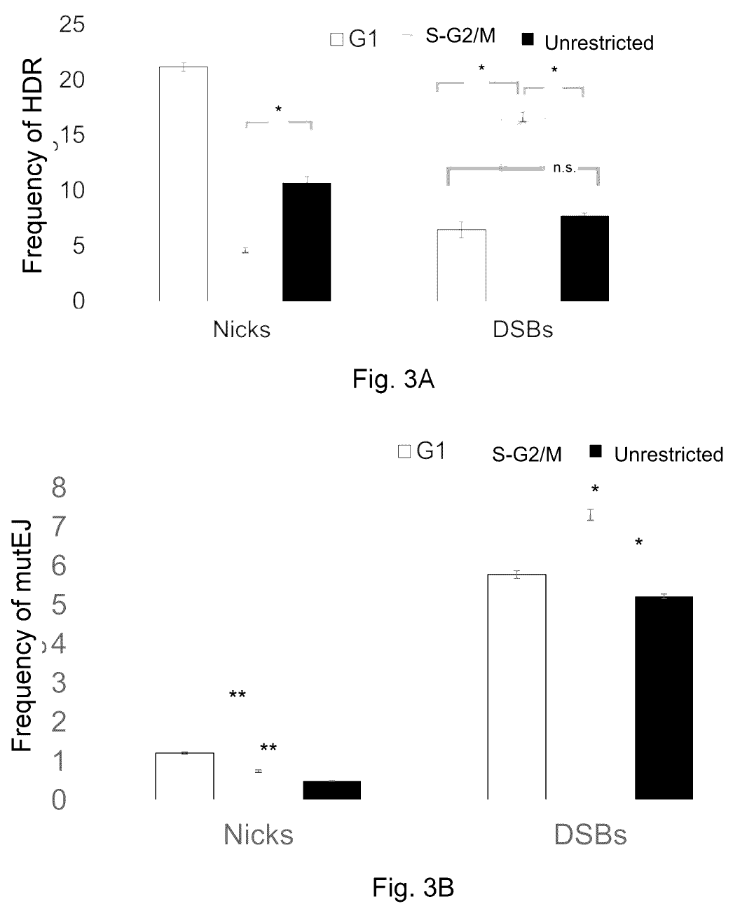

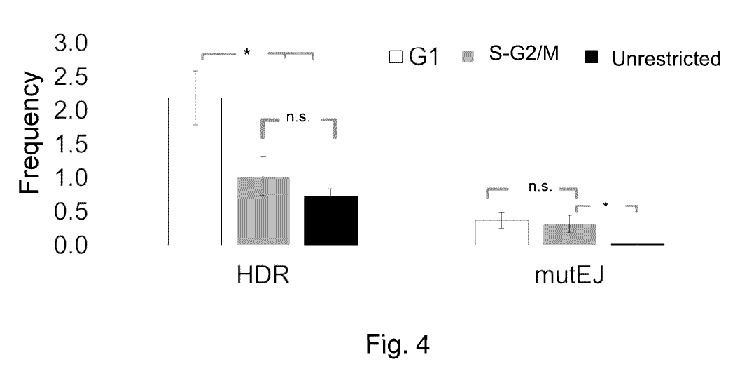

The frequency of HDR and mutEJ repair of nicks and DSBs throughout the cell cycles is depicted in FIGS. 3A-3B. The frequency of HDR and mutEJ repair at nicks throughout the cell cycle with cell cycle regulated Cas9 endonuclease depicted in FIG. 4.

The results provided herein demonstrate that improved safety and efficiency of gene correction by inducing a nick in G1 phase of the cell cycle. The HDR:mutEJ ratio increased to about 20:1 with nicks in G1 phase when compared to DSBs in S-G2/M phase with a ratio of about 2:1. Single stranded oligonucleotides serve as high efficiency donors at DSBs, but are accompanied by high mutEJ levels. The addition of a dominant negative BRCA2 peptide (BRC3) increased HDR levels by down-regulating the canonical HDR pathway and activating a highly efficient alternative HDR pathway (altHDR). altHDR may be of significant use for treating canonical HDR efficient tumors, such as ovarian and breast cancers with mutations in genes such as BRCA2 or RAD51. Restriction of CRISPR/Cas9 activity to non-cycling cells such as neurons can increase editing efficiency and minimize off-target mutations. Without wishing to be bound by theory, there may appear to be an increase in nick-initiated alternative HDR in G1 since there is no sister chromatid to undergo homologous pairing with which increases the likelihood that the GFP donor will be used. The Cas9 nickase is stabilized by the presence of the CDT1 cell cycle tag which can increase its efficacy.

### Example 3

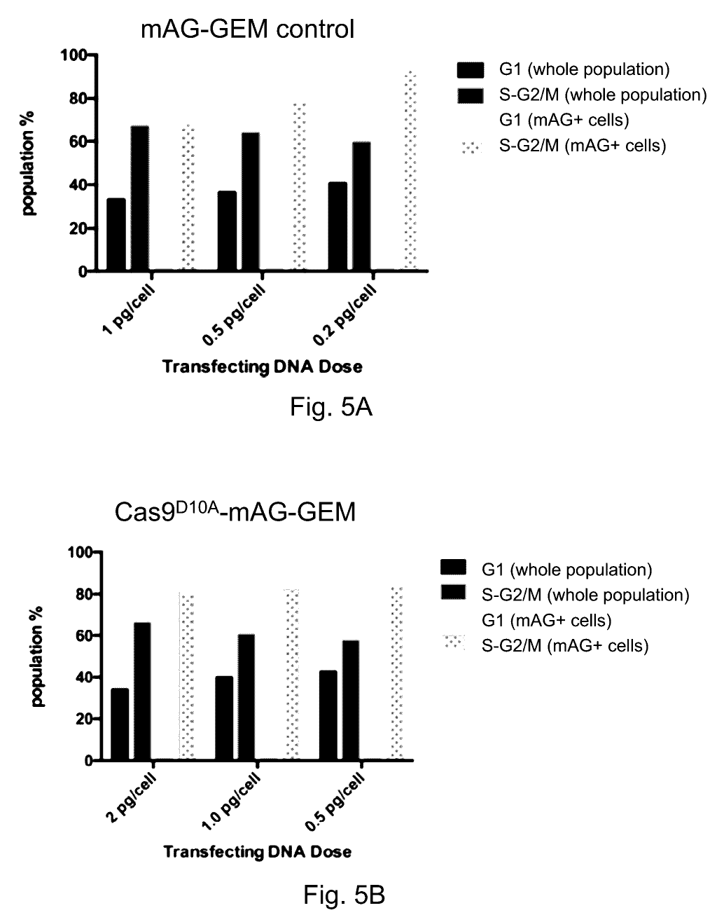

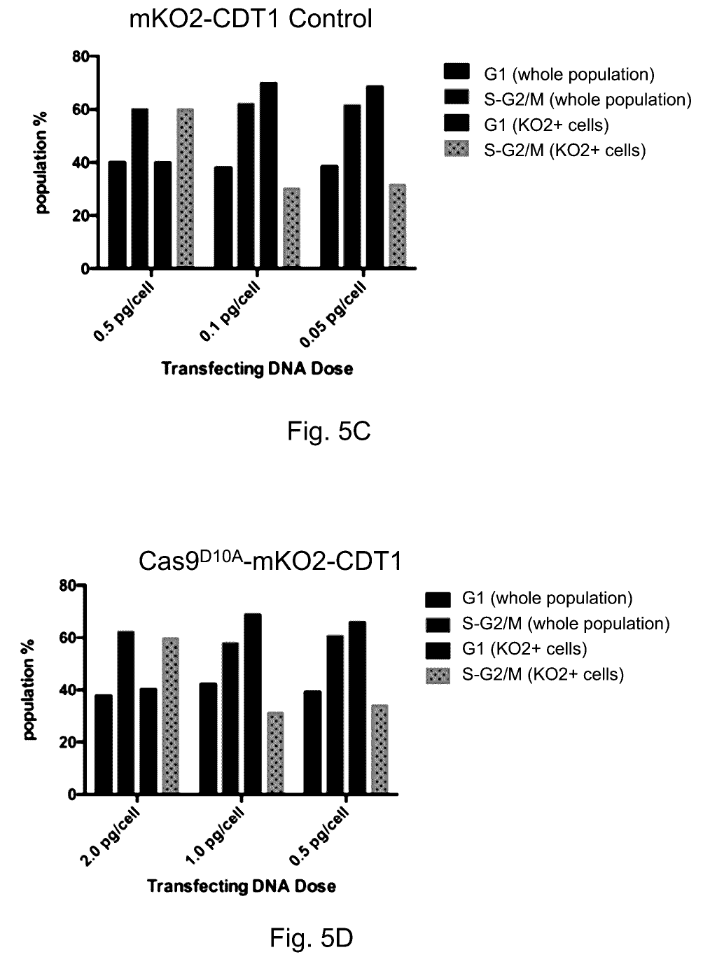

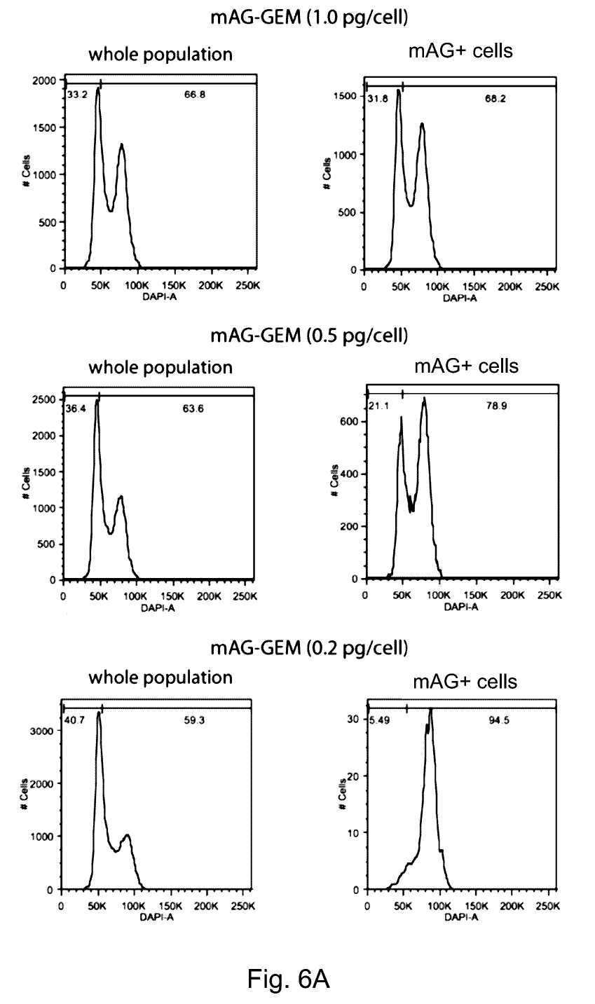

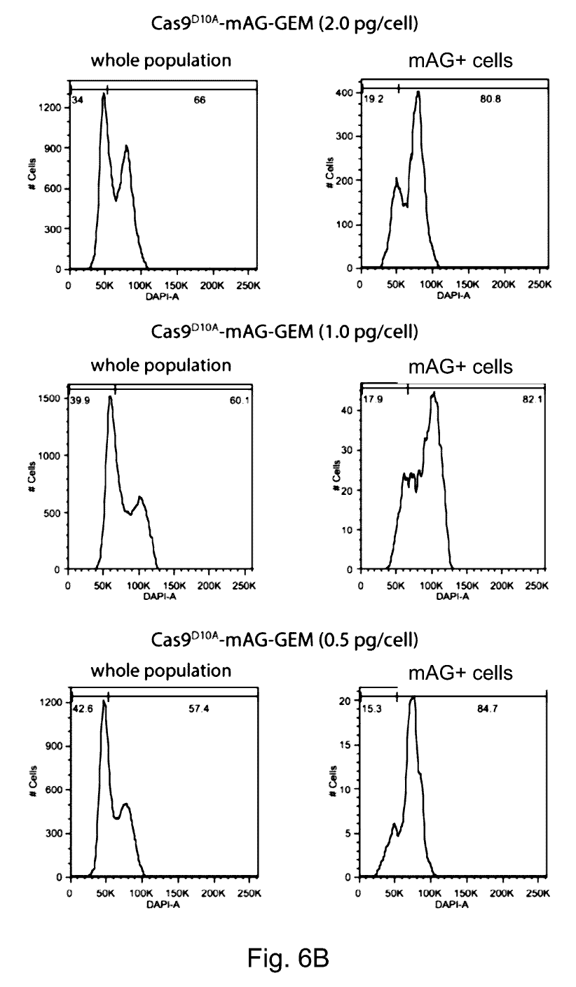

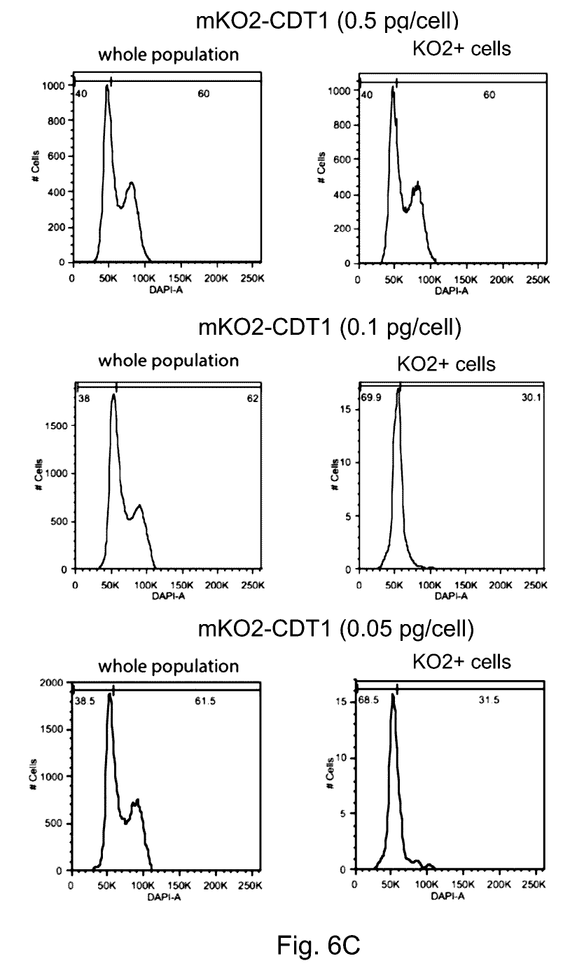

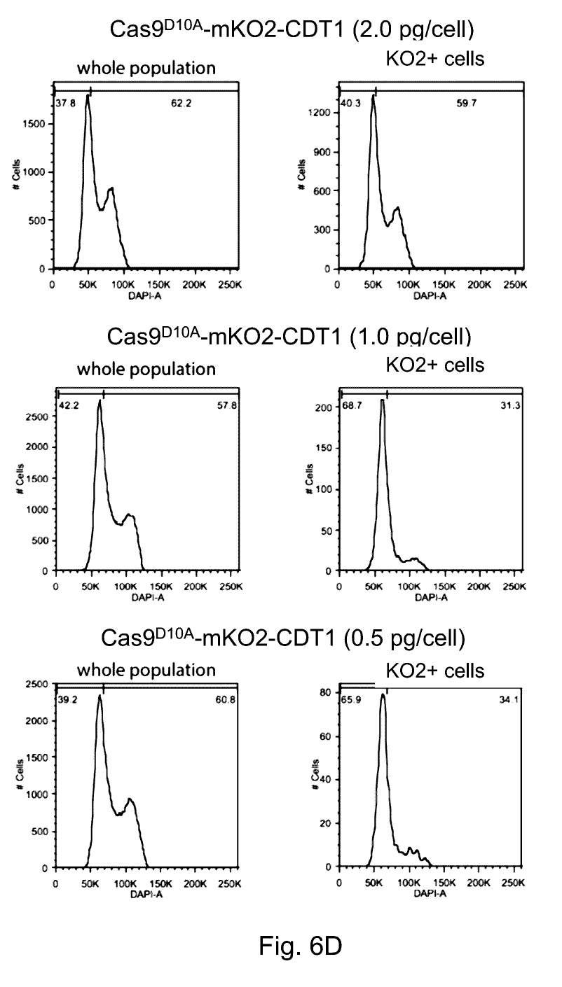

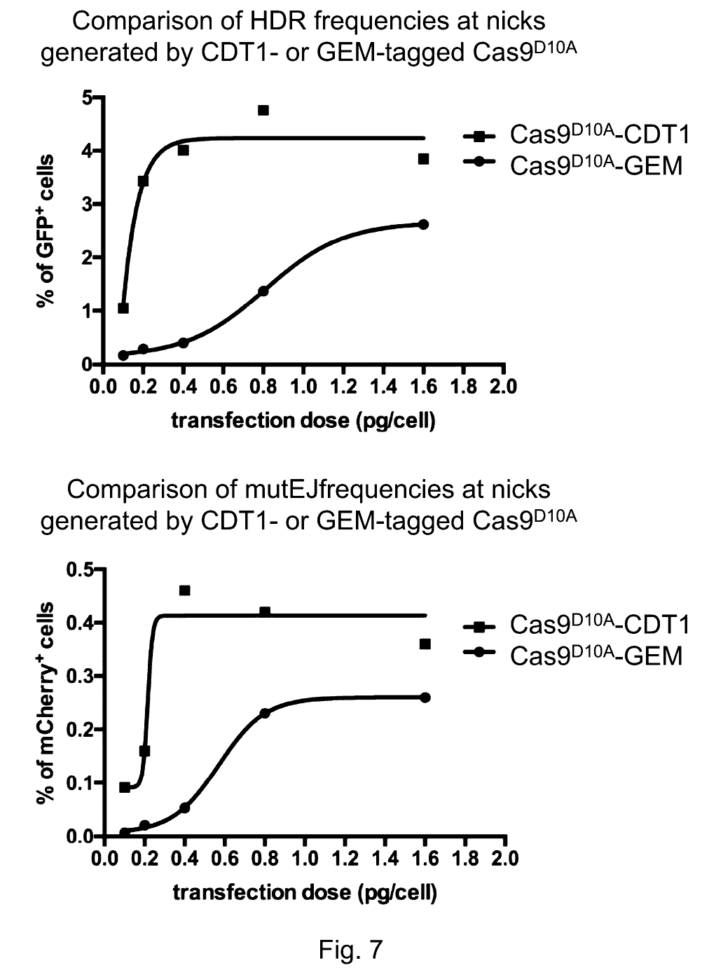

Demonstrated herein is cell cycle-specific expression of Cas9^{D10A} in 293T cells (FIGS. 5A-5D and 6A-6D). This cell cycle-specific expression resulted in preferential use of mutEJ and/or HDR as depicted in FIG. 7.

Table 10 presents data demonstrating the effect of restricting targeted nicks to G1 or S-G2/M phase of cell cycle in experiments that assay frequencies of homology-directed repair (HDR) and mutagenic end-joining (mutEJ) at a chromosomal Traffic Light reporter construct in human HEK 293T cells. Cells were treated with siBRCA2 to inhibit canonical HDR and promote alternative HDR. Targeted nicks were generated by Cas9^{D10A} bearing tags that restrict nuclear activity to G1 phase or S phase. In these conditions, HDR occurs with 11-fold greater efficiency at G1 phase nicks relative to S-G2/M phase nicks.

TABLE 10

| | Targeted | Cell cycle | HDR | mutEJ |
|---|---|---|---|---|
| Enzyme | break | phase | frequency | frequency |
| Cas9^{D10A}-CDT1 | Nick | G1 | 19% | 5.5% |
| Cas9^{D10A}-GEM | Nick | S-G2/M | 1.7% | 0.9% |

Table 12 presents data demonstrating the effect of restricting targeted DSBs to G1 or S phase of cell cycle in experiments that assay frequencies of homology-directed repair (HDR) and mutagenic end-joining (mutEJ) at a chromosomal Traffic Light reporter construct in human HEK 293T cells. Targeted DSBs were generated by Cas9 bearing tags that restrict nuclear activity to G1 phase or S-G2/M phase. HDR occurs with 3.5-fold greater efficiency at S-G2/M phase DSBs relative to G1 phase DSBs.

TABLE 12

| | | | HDR | mutEJ |
|---|---|---|---|---|
| Enzyme | Targeted break | Cell cycle phase | frequency | frequency |
| Cas9 | DSB | G1 and S-G2/M | 17.2% | 16.8% |
| Cas9-CDT1 | DSB | G1 | 4.3% | 13.1% |
| Cas9-GEM | DSB | S-G2/M | 14.9% | 13.4% |

Table 13 presents data demonstrating the effect of restricting targeted nicks to G1 or S-G2/M phases of cell cycle in experiments that assay frequencies of homology-directed repair (HDR) at the endogenous CD44 gene on chromosome 11 in human HT1080 cells. Cells were treated with siBRCA2 to inhibit canonical HDR and promote alternative HDR. Targeted nicks were generated by Cas9^{D10A} bearing tags that restrict nuclear activity to G1 phase or S-G2/M phases. HDR occurs with 16-fold greater efficiency at nicks generated in G1 phase relative to nicks generated in S-G2/M phases.

TABLE 13

| | | | HDR |
|---|---|---|---|
| Enzyme | Targeted break | Cell cycle phase | frequency |
| Cas9^{D10A} | Nick | G1 and S-G2/M | 11.1% |
| Cas9^{D10A}-CDT1 | Nick | G1 | 37.3% |
| Cas9^{D10A}-GEM | Nick | S-G2/M | 2.3% |

What is claimed herein is:

1. An engineered endonuclease comprising an endonuclease polypeptide and a cell cycle-dependent nuclear destruction tag.

2. The engineered endonuclease of claim 1, wherein the endonuclease polypeptide comprises a sequence-specific endonuclease.

3. The engineered endonuclease of claim 1, wherein the endonuclease polypeptide comprises an endonuclease selected from the group consisting of:
Cas9; a Cas9-derived nuclease; Cas9^{D10A}; a Cas9 nickase variant; a TALEN; a ZFN; Cpf1; a nuclease comprising a FokI cleavage domain; a RNA-guided engineered nuclease; and a homing endonuclease.

4. The engineered endonuclease of claim 1, wherein the cell cycle-dependent nuclear destruction tag comprises a sequence found in a protein selected from the group consisting of:
GEM; CDT1; Orc1; Cdc25A; Cyclin A; Cyclin B1; Securin; Plk1; Cdc6; Cyclin E; c-Jun; c-Myc; and RAG-2.

5. The engineered endonuclease of claim 1, wherein the cell cycle-dependent nuclear destruction tag comprises a Geminin (GEM) or chromatin licensing and DNA replication factor (CDT1) cell cycle-dependent nuclear destruction tag.

6. The engineered endonuclease of claim 4, wherein the cell cycle-dependent nuclear destruction tag is selected from SEQ ID NO: 4, SEQ ID NO: 6, or SEQ ID NOs: 8-12.

7. The engineered endonuclease of claim 1, wherein the tag is located at the C-terminus of the endonuclease.

8. The engineered endonuclease of claim 1, further comprising a linker sequence between the endonuclease polypeptide and cell cycle-dependent nuclear destruction tag.

9. The engineered endonuclease of claim 8, wherein the linker sequence comprises the sequence GGGGS (SEQ ID NO: 2).

10. An isolated nucleic acid molecule encoding the engineered endonuclease of claim 1.

11. A method of modifying the sequence of a target nucleic acid molecule, the method comprising:
contacting the target nucleic acid molecule with the engineered endonuclease of claim 1.

12. The method claim 11, wherein the target nucleic acid molecule is further contacted with a donor nucleic acid sequence.

13. The method claim 11, wherein the engineered endonuclease comprises a Cas9 or Cas9-derived endonuclease polypeptide and the method further comprises contacting the target nucleic acid molecule with one or more crRNA, tracrRNA, or sgRNA molecules.

14. The method of claim 11, wherein the engineered endonuclease comprises an endonuclease polypeptide and a G1-restricting cell cycle-dependent nuclear destruction tag and the modification thereby occurs via homology-directed repair.

15. The method of claim 17, wherein the G1-restricting cell cycle-dependent nuclear destruction tag is a CDT1 cell cycle-dependent nuclear destruction tag.

16. The method of claim 11, wherein the engineered endonuclease comprises an endonuclease polypeptide and a S-G2/M-restricting cell cycle-dependent nuclear destruction tag and the modification thereby occurs via non-homologous end-joining (NHEJ) or mutagenic end-joining (mutEJ).

17. The method of claim 16, wherein the S-G2/M-restricting cell cycle-dependent nuclear destruction tag is a GEM cell cycle-dependent nuclear destruction tag.


SEQUENCE LISTING (12 sequences)

SEQ ID NO: 1 - DNA, 99 bp, Artificial Sequence
  Description of Artificial Sequence Synthetic oligonucleotide
tggacggcgacgtaaacggccacaagttcagcgtgtccggcgagggtgagggcgatgccacctacggcaagctgaccctgaagttcatctgcaccaccg

SEQ ID NO: 2 - protein, 5 aa, Artificial Sequence
  Description of Artificial Sequence Synthetic peptide
GGGGS

SEQ ID NO: 3 - DNA, 2742 bp, Homo sapiens
cccgcctcttcctcccttccttctttccttgctttcgccgcgcactccgccgccatggagcagcgccgcgtcaccgacttcttcgcgcgccgccgccccgggcccccccgcatcgcgccgcccaagctggcctgccgcacccccagccccgccaggcccgcactccgcgccccggcctccgctaccagtggcagccgcaagcgcgcccgcccgcccgccgcccccggacgcgaccaggccaggccaccggcccgcaggagactgcggctgtcggtggacgaggtttccagccccagtacccccgaggccccagacatcccagcctgcccttctccgggccagaagataaagaaatccaccccggcagcaggtcagccgccccacctgacatccgcgcaggaccaggacaccatctctgagcttgcgtcatgcctgcaacgggcccgggagctgggggcaagagtccgggcgctgaaggccagtgcccaggatgctggggagtcctgcaccccagaggccgagggccgccctgaggagccatgtggcgagaaggcgcccgcctaccagcgcttccatgccctggcccagcccggcctgccgggactcgtgctgccctacaagtaccaggtgctggcggagatgttccgcagcatggacaccatcgtgggcatgctccacaaccgctccgagacgcccacctttgccaaggtccagcggggcgtccaggacatgatgcgtaggcgttttgaggagtgcaatgttggccagatcaaaaccgtgtacccggcctcctaccgcttccgccaggagcgcagtgtccccaccttcaaggatggcaccaggaggtcagattaccagctcaccatcgagccactgctggagcaggaggctgacggagcagccccccagctcacggcctcgcgcctcctgcagcgacggcagatcttcagccagaagctggtggagcatgtcaaggagcaccacaaggccttcctggcctccctgagccccgccatggtggtgccggaggaccagctgacccgctggcacccgcgcttcaacgtggatgaagtacccgacatcgagccggccgcgctgccccagccacccgccacggagaagctcaccactgctcaggaggtgctggcccgggcccgcaacctgatttcacccaggatggagaaggccttgagtcaattggccctgcgctctgctgcgcccagcagccccgggtctcccaggccagcactgccggctaccccaccagccaccccgcctgcagcctctcccagtgctctgaagggggtgtcccaggatctgctggagcggatccgagccaaggaggcacagaagcagctggcacagatgacgcggtgcccggagcaggagcagcggctgcagcgcttagaacggctgcctgagctggcccgcgtgctgcggagcgtctttgtgtccgaacgcaagcctgcgctcagcatggaggtggcctgtgccaggatggtgggcagctgttgtactatcatgagccctggggaaatggagaagcacctgctgctcctctccgagctgctgccggactggctcagcctccaccgcatccgcaccgacacctacgtcaagctggacaaggccgcggacctcgcccacatcactgcacgcctggcccaccagacacgtgctgaggaggggctgtgagcctgggggccactgtggacagacgtgggcttcagaagctcgctggcctgggcccaccagcattttcttttatgaacatgatacactttggccttcctttccccagcgcccctgagggccagaggcagatgtgggctgcaggctgcacagcccgagggtctctggctgcgggcggtgggccccttcatggggctcacctggtggattcacattaaaccggtttctgtgggcacctctgtccttgctgctggtggggaagggaagccagatccagcaccccctggggggccatcgggagtgtggctgggggtgaagggggctctgtggcaatatggggttgggtagtgtgggtggcaggccatcccctctaatcttggaacctctgaatatgggacctcccacagcaaagggtgacttttgtcattaagaaagactggggtgggtgtggtggctcacgcctgtaaccccagcactttgggaggccaaggtgggcagatcacgaggtcaagagatcgagaccatcctggcgaacatggtgaaaccccatctctactaaaaatacaaaaaattagccgggtgtggtggtgggcacctgtcgtcccagctactagggaggctgaggcaggagaatggtgtgaacccaggaggcacagcttgcagtgagcgaagatcgcaccactgcacgcactccagcctgggtgacagagcgagactccgtctcaaaaaaaaaaatttcaagactggagaggtgatcctgaattgtccagctacgccccatgtcatcacagggccttcatgacagggccagagccagccagctttgaagacgcggccctgccccgacacaggcagcctggagaagctgggcaggacaagtaggacatccctggagcctccagaagggactggcctctgcccacaccttgacttcagtatttctgacctcctaaactctaataaagtcatgcttacagccactaaaaaaaaaaaaaaaaaaaaaaaaaaaaaaaaaaaaaaaaaaaaaaaaaaaaaaaaaaaaaaaaaaaa

SEQ ID NO: 4 - protein, 91 aa, Homo sapiens
PSPARPALRAPASATSGSRKRARPPAAPGRDQARPPARRRLRLSVDEVSSPSTPEAPDIPACPSPGQKIKKSTPAAGQPPHLTSAQDQDTI

SEQ ID NO: 5 - DNA, 1275 bp, Homo sapiens
gtctgcgtcagttggtcacgtggttgttcggagcgggcgagcggagttagcagggctttactgcagagcgcgccgggcactccagcgaccgtggggatcagcgtaggtgagctgtggccttttgcgaggtgctgcagccatagctacgtgcgttcgctacgaggattgagcgtctccacccagtaagtgggcaagaggcggcaggaagtgggtacgcaggggcgcaaggcgcacagcctctagacgactcgctttccctccggccaacctctgaagccgcgtcctactttgacagctgcagggccgcggcctggtcttctgtgcttcaccatctacataatgaatcccagtatgaagcagaaacaagaagaaatcaaagagaatataaagaatagttctgtcccaagaagaactctgaagatgattcagccttctgcatctggatctcttgttggaagagaaaatgagctgtccgcaggcttgtccaaaaggaaacatcggaatgaccacttaacatctacaacttccagccctggggttattgtcccagaatctagtgaaaataaaaatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagagagactgaatggtgaacctctggataattttgaatcactggataatcaggaatttgattctgaagaagaaactgttgaggattctctagtggaagactcagaaattggcacgtgtgctgaaggaactgtatcttcctctacggatgcaaagccatgtatatgaaatgcattaatatttgactgttgagaattttactgccgaagtttacctccactagttctttgtagcagagtacataactacataatgccaactctggaatcaaatttccttgtttgaatcctgggaccctattgcattaaagtacaaatactatgtatttttaatctatgatggtttatgtgaataggattttctcagttgtcagccatgacttatgtttattactaaataaacttcaaactcctgttgaacattgtgtataacttagaataatgaaatataaggagtatgtgtagaaaaaaaaaa

SEQ ID NO: 6 - protein, 110 aa, Homo sapiens
MNPSMKQKQEEIKENIKNSSVPRRTLKMIQPSASGSLVGRENELSAGLSKRKHRNDHLTSTTSSPGVIVPESSENKNLGGVTQESFDLMIKENPSSQYWKEVAEKRRKAL

SEQ ID NO: 7 - DNA, 3053 bp, Homo sapiens
gagcgcggctggagtttgctgctgccgctgtgcagtttgttcaggggcttgtggtggtgagtccgagaggctgcgtgtgagagacgtgagaaggatcctgcactgaggaggtggaaagaagaggattgctcgaggaggcctggggtctgtgaggcagcggagctgggtgaaggctgcgggttccggcgaggcctgagctgtgctgtcgtcatgcctcaaacccgatcccaggcacaggctacaatcagttttccaaaaaggaagctgtctcgggcattgaacaaagctaaaaactccagtgatgccaaactagaaccaacaaatgtccaaaccgtaacctgttctcctcgtgtaaaagccctgcctctcagccccaggaaacgtctgggcgatgacaacctatgcaacactccccatttacctccttgttctccaccaaagcaaggcaagaaagagaatggtccccctcactcacatacacttaagggacgaagattggtatttgacaatcagctgacaattaagtctcctagcaaaagagaactagccaaagttcaccaaaacaaaatactttcttcagttagaaaaagtcaagagatcacaacaaattctgagcagagatgtccactgaagaaagaatctgcatgtgtgagactattcaagcaagaaggcacttgctaccagcaagcaaagctggtcctgaacacagctgtcccagatcggctgcctgccagggaaagggagatggatgtcatcaggaatttcttgagggaacacatctgtgggaaaaaagctggaagcctttacctttctggtgctcctggaactggaaaaactgcctgcttaagccggattctgcaagacctcaagaaggaactgaaaggctttaaaactatcatgctgaattgcatgtccttgaggactgcccaggctgtattcccagctattgctcaggagatttgtcaggaagaggtatccaggccagctgggaaggacatgatgaggaaattggaaaaacatatgactgcagagaagggccccatgattgtgttggtattggacgagatggatcaactggacagcaaaggccaggatgtattgtacacgctatttgaatggccatggctaagcaattctcacttggtgctgattggtattgctaataccctggatctcacagatagaattctacctaggcttcaagctagagaaaaatgtaagccacagctgttgaacttcccaccttataccagaaatcagatagtcactattttgcaagatcgacttaatcaggtatctagagatcaggttctggacaatgctgcagttcaattctgtgcccgcaaagtctctgctgtttcaggagatgttcgcaaagcactggatgtttgcaggagagctattgaaattgtagagtcagatgtcaaaagccagactattctcaaaccactgtctgaatgtaaatcaccttctgagcctctgattcccaagagggttggtcttattcacatatcccaagtcatctcagaagttgatggtaacaggatgaccttgagccaagaaggagcacaagattccttccctcttcagcagaagatcttggtttgctctttgatgctcttgatcaggcagttgaaaatcaaagaggtcactctggggaagttatatgaagcctacagtaaagtctgtcgcaaacagcaggtggcggctgtggaccagtcagagtgtttgtcactttcagggctcttggaagccaggggcattttaggattaaagagaaacaaggaaacccgtttgacaaaggtgtttttcaagattgaagagaaagaaatagaacatgctctgaaagataaagctttaattggaaatatcttagctactggattgccttaaattcttctcttacaccccacccgaaagtattcagctggcatttagagagctacagtcttcattttagtgctttacacattcgggcctgaaaacaaatatgaccttttttacttgaagccaatgaattttaatctatagattctttaatattagcacagaataatatctttgggtcttactatttttacccataaaagtgaccaggtagaccctttttaattacattcactacttctaccacttgtgtatctctagccaatgtgcttgcaagtgtacagatctgtgtagaggaatgtgtgtatatttacctcttcgtttgctcaaacatgagtgggtatttttttgtttgttttttttgttgttgttgtttttgaggcgcgtctcaccctgttgcccaggctggagtgcaatggcgcgttctctgctcactacagcacccgcttcccaggttgaagtgattctcttgcctcagcctcccgagtagctgggattacaggtgcccaccaccgcgcccagctaattttttaatttttagtagagacagggttttaccatgttggccaggctggtcttgaactcctgaccctcaagtgatctgcccaccttggcctccctaagtgctgggattataggcgtgagccaccatgctcagccattaaggtattttgttaagaactttaagtttagggtaagaagaatgaaaatgatccagaaaaatgcaagcaagtccacatggagatttggaggacactggttaaagaatttatttctttgtatagtatactatgttcatggtgcagatactacaacattgtggcattttagactcgttgagtttcttgggcactcccaagggcgttggggtcataaggagactataactctacagattgtgaatatatttattttcaagttgcattctttgtctttttaagcaatcagatttcaagagagctcaagctttcagaagtcaatgtgaaaattccttcctaggctgtcccacagtctttgctgcccttagatgaagccacttgtttcaagatgactactttggggttgggttttcatctaaacacatttttccagtcttattagataaattagtccatatggttggttaatcaagagccttctgggtttggtttggtggcattaaatgg

SEQ ID NO: 8 - DNA, 8 bp, Homo sapiens
ggatcctg

SEQ ID NO: 9 - protein, 12 aa, Homo sapiens
QRRVTDFFARRR

SEQ ID NO: 10 - protein, 20 aa, Unknown
  Description of Unknown G1-restricting cell cycle-dependent nuclear destruction tag of RAG2
QTPKRNPPLQKPPMKSLHKK

SEQ ID NO: 11 - protein, 9 aa, Artificial Sequence
  Description of Artificial Sequence Synthetic peptide
RXXLXXXXN

SEQ ID NO: 12 - protein, 7 aa, Artificial Sequence
  Description of Artificial Sequence Synthetic peptide
KENXXXN